SEQUENCE LISTING (5 sequences)

SEQ ID NO: 1 - protein, 222 aa, synthetic construct
MFGGAPVKVFGPAMSTNVARVTLCLEEVGAEYEVVNIDFNTMEHKSPEHLARNPFGQIPAFQDGDLLLWESRAISKYVLRKYKTDEVDLLREGNLKEAAMVDVWTEYDAHTYNPALSPIVYECLINPLMRGLPTNQTVVDESLEKLKKVLEVYEARLSQHKYLAGDFVSFADLNHFPYTFYFMATPHAALFDSYPHVKAWWDRLMARPAVKKIAATMVPPKA

SEQ ID NO: 2 - protein, 222 aa, synthetic construct
MFGGAPVKVFGPAMSTNVARVTLCLEEVGAEYEVVDIDFKAMEHKSPEHLVRNPFGQIPAFQDGDLLLFESRAIAKYVLRKYKTDEVDLLREGNLKEAAMVDVWTEYDAHTYNPALSPIVYECLINPLMRGLPTNQTVVDESLEKLKKVLEVYEARLSQHKYLAGDFVSFADLNHFPYTFYFMATPHAALFDSYPHVKAWWDRLMARPAVKKIAATMVPPKA

SEQ ID NO: 3 - DNA, 32 bp, synthetic construct
  source 1..32: mol_type = other DNA
atgtttggaggagcacctgtaaaagtctttgg

SEQ ID NO: 4 - DNA, 27 bp, synthetic construct
  source 1..27: mol_type = other DNA
atgtatatctccttcttatagttaaac

SEQ ID NO: 5 - DNA, 18 bp, synthetic construct
  source 1..18: mol_type = other DNA
tttggaggatttggagga

(19) United States
(12) Patent Application Publication
(10) Pub. No.: US 2026/0153494 A1
(43) Pub. Date: Jun. 4, 2026

(54) CATLYTICALLY MODIFIED ENZYME-BASED BIOSENSORS AND METHODS OF PRODUCTION THEREOF

(71) Applicant: AGRICUL TURAL UNIVERSITY OF ATHENS, Athens (GR)

(72) Inventors: Nikolaos Labrou, Athens (GR); Elisavet Ioannou, Athens (GR)

(21) Appl. No.: 19/498,485

(22) PCT Filed: Jul. 17, 2023
(86) PCT No.: PCT/EP2023/069803

§ 371 Date: Feb. 17, 2026

(51) Int. Cl. G01N 33/18; C12N 11/10; C12Q 1/48

(52) U.S. Cl. CPC G01N 33/184; C12N 11/10; C12Q 1/48; G01N 2333/91177

(57) ABSTRACT

The present invention relates to catalytically modified enzyme-based biosensors comprising supramolecular protein assemblies of glutathione S-transferase (GST) and methods of production thereof. Further, the invention relates to applications of said biosensors in detecting and determining the presence of pollutants, preferably herbicides in water and kits suitable for such detection.

### FIELD OF THE INVENTION

The present invention relates to catalytically modified enzyme-based biosensors comprising supramolecular protein assemblies of glutathione S-transferase (GST) and methods of production thereof. Further, the invention relates to applications of said biosensors in detecting and determining the presence of pollutants, preferably herbicides in water and kits suitable for such detection.

### BACKGROUND

Glutathione S-transferases (GSTs) are a family of multifunctional enzymes that play a crucial role in the cellular detoxification against exogenous and noxious molecules, including drugs and environmental pollutants. GSTs are able to conjugate glutathione (GSH) to a wide range of hydrophobic and electrophilic compounds, making them more soluble and less toxic ((Labrou et al., 2015; Nianiou-Obeidat et al., 2017; Perperopoulou et al., 2018; Board & Menon, 2013). GSTs are widely distributed in nature and they are found in both eukaryotic and prokaryotic organisms. Cytosolic GSTs are homodimer enzymes and each subunit consists of two structural domains. The N-terminal domain includes most of the GSH-binding site (G-site) which exhibits a highly conserved thioredoxin motif. The C-terminal domain consists exclusively of α-helices that contribute to the binding of electrophilic substrates (H-site) (Sylvestre-Gonon et al, 2019; Georgakis et al., 2020; Ioannou et al., 2022). The plant cytosolic phi (GSTFs) and tau (GSTUs) class GSTs display high substrate specificity and are found to assist the cell detoxification of pesticides (Dixon et al., 2002a). In addition, they have been linked to the phenomenon of multiple herbicide resistance (MHR), which is associated with enhanced metabolism and detoxification properties of endogenous enzymes found in numerous weeds (Cummins et al., 2013; Nakka et al., 2019; Georgakis et al., 2020; Ioannou et al., 2022). GSTs have been successfully utilized for the development of biosensors with several applications, such as detection of pesticides whose residues in the environment can cause long-term damage to human health, given their versatility in catalysing different reactions and in binding several substrates and inhibitors (Bocedi et al., Nutrients 2019, 11, 1741). Traditionally, the detection of such pesticides or insecticides is performed with gas or liquid chromatography and mass spectrometry (Del Buono et al., 2005; Xu et al., 2007). For instance, gas chromatography is the most commonly used method for butachlor determination (Lei et al., 2022). Such techniques allow for detection at concentrations as low as 5 ng/L with excellent yield (Yakovleva et al., 2003). However, they require specialized and expensive equipment, thus may not be considered ideal for screening of large sample populations. Therefore, GST based biosensors provide an alternative solution to standard analytical chromatographic methods allowing for flexible (e.g. in situ), rapid and low cost detection of environmental pollutants such as pesticides or insecticides.

It is, thus, an aim of the invention to provide such optimised GST based biosensors, which are more efficient in terms of sensitivity, cost and application flexibility. Additionally, the present invention provides methods of production of such optimised GST based biosensors.

### SUMMARY

The present invention relates to catalytically modified enzyme-based biosensors comprising supramolecular protein assemblies of glutathione transferase (GST) and methods of production thereof. Further, the invention relates to applications of said biosensors in detecting and determining the presence of xenobiotics such as pollutants, preferably herbicides in a sample such as a water sample, and kits for such detection.

In one aspect the present invention relates to a biosensor comprising a glutathione S-transferase homohexamer, wherein each transferase monomer of said homohexamer, comprises a Phenylalanine-Glycine-Glycine tag at its N-terminal site, wherein said N-tagged monomer is assembled to a homohexamer through binding of Cucurbit[n]uril to said Phenylalanine-Glycine-Glycine tag and wherein said homohexamer is further characterised that it is immobilised on a polymer carrier.

In one embodiment said biosensor is characterized in that said N-tagged monomer is assembled to a homohexamer through binding of Cucurbit[8]uril to said Phenylalanine-Glycine-Glycine tag and the concentration ratio of said N-tagged oligomer to Cucurbit[8]uril is 1:1.

In one embodiment said glutathione S-transferase monomer of said biosensor comprises the SEQ ID NO: 1 or SEQ ID NO: 2.

In one embodiment said homohexamer of said biosensor is immobilized on a glutaraldehyde-crosslinked chitosan carrier.

In another aspect the invention relates to a method of production of a biosensor of the invention characterized in that said method comprises the following steps of
mixing a chitosan gel with a glutaraldehyde solution so that a cross-linked chitosan carrier is formed
adding a solution of said glutathione S-transferase homohexamer to said cross-linked chitosan carrier.

In one embodiment said method further comprises the following steps of
dissolving 2%-4% (w/v) of chitosan in 2% (v/v) acetic acid so that a chitosan gel is formed
storing said chitosan gel for 12 to 24 hours at 4° C.
adding a glutaraldehyde solution at a concentration of 0.4%-1% (v/v), said glutaraldehyde being diluted in 20 mM KH₂PO₄, PH 7.0
washing with 2 mL 20 mM KH₂PO₄, PH 7
storing the cross-linked chitosan carrier for 3 to 10 days
adding a solution of glutathione S-transferase homohexamer at a concentration of 0.5 to 1 mg/mL
incubating said solution added to the carrier for 12 to 24 hours at around 25° C. with stirring at about 50-80 rpm
removing the non-absorbed solution of glutathione S-transferase homohexamer
incubating the carrier with a solution of 2 mg/mL NaBH₄ for about 30 min
washing with 20 mM KH₂PO₄, PH 7 in order to remove the unbound enzyme

In one embodiment said method further comprises the following steps of
dissolving 2%-4% (w/v) of chitosan in 2% (v/v) acetic acid so that a chitosan gel is formed
storing said chitosan gel for 12 to 24 hours at 4° C.
adding a glutaraldehyde solution at a concentration of 0.4%-1% (v/v), said glutaraldehyde being diluted in 20 mM KH₂PO₄, PH 7.0
washing with 2 mL 20 mM KH₂PO₄, PH 7
storing the cross-linked chitosan carrier for 3 to 10 days
adding a solution of glutathione S-transferase homohexamer at a concentration of 0.5 to 1 mg/mL
incubating said solution added to the carrier for 12 to 24 hours at around 25° C. with stirring at about 50-80 rpm
removing the non-absorbed solution of glutathione S-transferase homohexamer
incubating the carrier with a solution of 2 mg/mL NaBH₄ for about 30 min
washing with 20 mM KH₂PO₄, PH 7 in order to remove the unbound enzyme

In a further aspect the invention relates to a method of detecting a xenobiotic in a sample using a biosensor of the present invention

In one embodiment said xenobiotic of said method is a herbicide, preferably a chloroacetanilide herbicide, more preferably butachlor and said sample is a water sample.

In one other aspect, the invention relates to a use of the biosensor of the invention for the detection of a xenobiotic, preferably a chloroacetanilide herbicide, more preferably butachlor, in a sample.

In an even further aspect the invention relates to a kit comprising a biosensor of the invention for the detection of a xenobiotic, preferably a chloroacetanilide herbicide, more preferably butachlor, in a sample.

### BRIEF DESCRIPTION OF THE DRAWINGS

FIG. 1 (A-H): Steady-state kinetic analysis using GSH as a variable substrate and CDNB at a fixed concentration. FIG. 1B, FIG. 1D, FIG. 1F and FIG. 1H represent enzymes FGGsh101, FGGsh101:CB[8], FGGsh155 and FGGsh101:CB[8] respectively, using the CDNB as a variable substrate. FIG. 1A, FIG. 1C, FIG. 1E and FIG. 1G. represent enzymes FGGsh101, FGGsh101:CB[8], FGGsh155 and FGGsh101:CB[8] respectively, using GSH at a fixed concentration [B: (I-IV)]. The measurements were performed in triplicate.

FIG. 2 (A-D): Remaining fluorescence intensity (%) of the FITC-labeled FGG-GSTFs after addition of CB[8] in 1:1 (15 μM of CB[8] to 15 μM enzyme) and 2:1 (7.5 M of CB[8] to 15 μM enzyme) ratios. Remaining fluorescence intensity (%) in FITC labelled FGGSH101 (FIG. 2A) and FGGsh155 (FIG. 2B) at pH 9 for selective binding at lysine residues. Remaining fluorescence intensity (%) in FITC labelled FGGSH101 (FIG. 2C) and FGGsh155 (FIG. 2D) at pH 6.5 for selective binding at cysteine residues. 100% fluorescence intensity corresponds to the labelled enzyme in absence of CB[8].

FIG. 3 (A-D): Remaining specific activity (%) of FITC labelled enzymes at pH 9 in the presence of CB[8]: FIG. 3A. FGGsh101:CB[8] (1:1), FIG. 3B. 2FGGsh101:CB[8] (2:1), FIG. 3C. FGGsh155:CB[8] (1:1), FIG. 3D. 2FGGsh155:CB[8] (2:1). 100% is defined by the specific activity on the day of CB[8] addition. Square shape including line indicates the remaining specific activity of the control reaction, where CB[8] was absent. Circle shape including line shows the remaining specific activity of FITC labelled enzymes in presence of CB[8]. Measurements were carried in triplicate.

FIG. 4 (A-D): Remaining specific activity (%) of FITC labelled enzymes at pH 6.5 in the presence of CB[8]: FIG. 4A. FGGsh101:CB[8] (1:1), FIG. 4B. 2FGGsh101:CB[8] (2:1), FIG. 4C. FGGsh155:CB[8] (1:1), FIG. 4D. 2FGGsh155:CB[8] (2:1). 100% is defined as the specific activity on the day of CB[8] addition. Square shape including line indicates the remaining specific activity of the control reaction, where CB[8] was absent. Circle shape including line shows the remaining specific activity of FITC labelled enzymes in the presence of CB[8]. Measurements were carried in triplicate.

FIG. 5 (A-E): Confocal laser scanning microscopy (CLSM) images of the FITC-labeled FGG-GSTFs at pH 9 (binding to ε-amines) on the 9th day after addition of CB[8]. The supernatant of each sample is shown in the first column and the precipitate in the second column: FIG. 5A. FGGsh101:CB[8], FIG. 5B. 2 FGGsh101:CB[8], FIG. 5C. FGGsh155:CB[8], FIG. 5D. 2 FGGsh155:CB[8] and FIG. 5E. standard image of a “control” reaction without CB[8].

FIG. 6 (A-D): Confocal laser scanning microscopy (CLSM) images of the FITC-labeled FGG-GSTFs at pH 9 (binding to ε-amines) on the 9th day after addition of CB[8]. The supernatant of each sample is shown in the first column and the precipitate in the second column: FIG. 6A. FGGsh101:CB[8], FIG. 6B. 2 FGGsh101:CB[8], FIG. 6C. FGGsh155:CB[8] and FIG. 6D. 2 FGGsh155:CB[8].

FIG. 7 (A-F): First derivative graphs for Tₘ determination of FGGsh101 after addition of C [8] at 1:1 (first column) and 2:1 (second column) stoichiometry. The graphs correspond to the day of addition Day 0, the 6th and the 16th day after it. FIG. 7A. 1:1, day 0, FIG. 7B 2:1, day 0. FIG. 7C. 1:1, day 6, FIG. 7D 2:1, day 6. FIG. 7E. 1:1, day 16, FIG. 7F 2:1, day 16. The peak Tₘ₀ corresponds to the melting point temperature of a “control” reaction without CB[8], Tₘ₁ and Tₘ₂ correspond to the melting points after the addition of CB[8].

FIG. 8 (A-F): First derivative graphs for Tₘ determination of FGGsh155 after addition of C [8] at 1:1 (first column) and 2:1 (second column) stoichiometry. The graphs correspond to the day of addition Day 0, the 6th and the 16th day after it. FIG. 8A. 1:1, day 0, FIG. 8B 2:1, day 0. FIG. 8C. 1:1, day 6, FIG. 8D 2:1, day 6. FIG. 8E. 1:1, day 16, FIG. 8F 2:1, day 16. The peak Tₘ₀ corresponds to the melting point temperature of a “control” reaction without CB[8], Tₘ₁ and Tₘ₂ correspond to the melting points after the addition of CB[8].

FIG. 9 (A-D): Indicative graphs of the first derivative of sh101 and sh155 without the FGG tag, on third day after the addition of CB[8] in stoichiometry of 1:1 and 2:1 enzyme to host. FIG. 9A. sh101 in stoichiometry of 1:1, FIG. 9B sh101 in stoichiometry of 2:1, FIG. 9C. sh155 in stoichiometry of 1:1, FIG. 9D. sh155 in stoichiometry of 2:1.

FIG. 10 (A-B): HPLC profile of FGGsh101 after addition of CB[8] in 1:1 ratio. FIG. 10A. Control reaction with absence of CB[8] and FIG. 10B. Sample of 15 μM FGGsh101 and 15 μM CB[8].

FIG. 11 (A-B): HPLC profile of FGGsh155 after addition of CB[8] in 1:1 ratio. FIG. 11A. Control reaction without CB[8] and FIG. 11B. Sample of 15 μM FGGsh155 and 15 μM CB[8].

FIG. 12 (A-B): Images of the “control” sample FGGsh155 without CB[8] derived by scanning electron microscopy (SEM).

FIG. 13 (A-E): Images of FGGsh155:CB[8] derived by scanning electron microscopy (SEM). White arrows show a few fine linear structures that very likely correspond to linear protein nanostructures (Li et al., 2017; Hou et al., 2013) and formations of 3-4 μm length and 1 μm width, which possibly correspond to previously detected formations of FITC-labeled enzymes by CLSM.

FIG. 14 (A-E): Crystal structure of FGGsh155:CB[8] hexamer. FIG. 14A-FIG. 14B: Ribbon representation of the hexamer protein. FIG. 14C. Secondary structure of the FGG-GSFT monomer, where CB[8]'s cavity is occupied by the N-terminal methionine and phenylalanine. The α-helixes (red) and β-strands (blue) are depicted. FIG. 14D. Surface hydrophobicity of the protein. Hydrophilic areas are colored in a blue range and hydrophobic areas in an orange/red range. Depictions were made by UCSF Chimera (Pettersen et al., 2004). FIG. 16E. Electrostatic surface of the hexamer as created by PyMol (Schrödinger & DeLano, 2020).

FIG. 15 (A-B): Superposition of the crystal structures sh155 (PDB code: 7ZA4) and FGGsh155: CB [8]. A. Sh155 subunit A and FGGsh155:cb[8] subunit E and B. Sh155 subunit B and FGGsh155:CB[8] subunit C. Imaging via UCSF Chimera (Pettersen et al., 2004).

FIG. 16 (A-B): Normal mode analysis of FGGsh155:CB[8]. FIG. 16A. Plot of deformation energy and FIG. 16B. Atomic fluctuation of the GSTF hexamer. The plots were produced by DynaMut (Rodrigues et al., 2018). The magnitude of deformation/fluctuation is depicted with thin to thick tubes (thin tubes represent lower deformation/fluctuation and thick tubes higher)

FIG. 17: The CB[8] triangular formation. Each CB[8] cavity hosts the first Met and the Phe side chain of the FGG-N-terminal tag. Depiction was created by PyMol (Schrödinger & DeLano, 2020).

FIG. 18: The entry of phenylalanine methionine into the cavity of the CB[8], as obtained from the crystal structure of the enzyme sample FGGsh155:CB[8].

FIG. 19: Interactions between the outer surface of CB[8] and specific residues of the binding subunit.

FIG. 20 (A-B): Steady-state kinetic analysis of the immobilized FGGsh155:CB[8]. FIG. 20A. Steady-state kinetic analysis with varying concentrations of GSH substrate and fixed CDNB concentration at 1 mM. FIG. 20B. Steady-state kinetic analysis with varying concentrations of CDNB and fixed GSH concentration at 2.5 mM. Measurements were carried out three times.

FIG. 21 (A-B): Dose-response inhibition curves of FGGsh155:CB[8] due to the herbicide butachlor for the determination of IC50 value. FIG. 21A. before and FIG. 21B. after immobilization. The data represent the mean±SD (N=3).

FIG. 22 (A-J): Kinetic inhibition analysis of the dimer enzyme sh155 (FIG. 22A, 22C, 22E, 22G, 22I) and the hexamer FGGsh155:CB[8] (FIGS. 22B, 22D, 22F, 22H, 22J). A. & B. Lineweaver-Burk plots for the inhibition of the enzymes by different concentrations of butachlor, at constant concentration of GSH and variable concentration of CDNB. C. & D. Secondary (derivative) graph. The slope values were derived from the Lineweaver-Burk graphs (A) & (B). The intersection point with the x axis corresponds to Kᵢ. E. & F. Secondary (derivative) graph where the data are derived from the Lineweaver-Burk graphs (A) & (B). The intersection point with the x axis corresponds to K. G. & H. Lineweaver-Burk plots for the inhibition of the enzymes by different concentrations of butachlor, at constant concentration of CDNB and variable concentration of GSH. I. & I. Secondary (derivative) graph. The slope values were derived from the Lineweaver-Burk graphs G & J, and the intersection point with the x axis corresponds to Kᵢ.

FIG. 23 (A-D): FIG. 23A. Linear correlation between the remaining activity of the immobilized FGGsh155:CB[8] and the butachlor concentration from 0 to 600 nM. FIG. 23B. Standard curves of the remaining activity (%) induced by different amounts of butachlor, determined in Athens' water supply network and in bottled mineral water samples. Recovery experiments using spiked water samples from the supply network (FIG. 23C) and bottled mineral water (FIG. 23D) with known amounts of butachlor. The concentrations of found butachlor were calculated based on the standard reference curves. Plots were obtained from at least three replicate measurements (Mean±SD, N=3).

FIG. 24: Remaining activity (%) after storage at 4° C. of the dimer GSTF enzyme (FGGsh155) and the hexamer FGGsh155:CB[8] before and after immobilization.

### DETAILED DESCRIPTION

The intensification of agricultural activities has resulted to the extensive use of pesticides that can be considered as pollutants when they are detected out of the controlled cultivation system (Sharma et al., 2015; Nehra et al., 2021; Gong et al., 2022). The European Union defines the maximum acceptable concentration of a pesticide in surface water at 1.0 μg/L and in drinking water at 0.1 μg/L per pesticide and up to 0.5 μg/L in total (Council Directive 98/83/EC).

Chloroacetanilide pesticides, namely acetochlor, alachlor, butachlor, metazachlor, metolachlor and propachlor, are widely used in agricultural practices (Abigail et al., 2015; Aladaghlo et al., 2016; Yu et al., 2023). They function mostly by inhibiting pathways in the biosynthesis of lipids, alcohols, fatty acids, proteins, isoprenoids or flavonoids (Heydens et al., 2002). Butachlor (2-chloro-2,6-diethyl-N-butoxymethyl-acetanilide) is a systemic and selective herbicide that is widely used in cultivation of rice, wheat, soybean and other crops (Dwivedi et al. 2012; Li et al., 2019), for the control of annual and broadleaf weeds (Wang et al. 2013; Lin et al., 2021). Butachlor inhibits enzymes in the biosynthesis of lipids and it also negatively affects several metabolic processes as well as the redox homeostasis (Gotz & Boger, 2004; Zhu et al., 2014). Inappropriate water management or rainfall usually results to its runoff into aquatic ecosystems (Ok et al., 2012; El-Nahhal & El-Nahhal, 2021). The half-life of butachlor is estimated up to 2.5 days in water (Huarong et al. 2010) and 2.67-30 days in soil depending on the conditions (Kaur et al. 2017; Mohanty & Jena 2019). Human exposure to butachlor may lead to adverse reactions in the gastrointestinal, neurological, cardiovascular, respiratory and/or immune system (Lo et al., 2008; Zhu et al., 2022b). In addition, it is considered a possible carcinogen and causes a dose-dependent increase in the frequency of chromosomal abnormalities in human lymphocytes (Sinha et al., 1995).

It is a subject of the present disclosure to provide a sensitive enzyme biosensor for the detection of environmental xenobiotics, such as pollutants, preferably chloroacetanilide pesticides. The enzyme biosensor of the invention comprises a supramolecular complex based on GST and utilises the ability of GST to form protein nanomaterials due to metal coordination (Bai et al., 2013; Zhang et al., 2012) or host-guest interactions induced by cucurbit[8]uril (CB[8]) (Wang et al., 2017; Hou et al., 2013). More particularly, said enzyme complex of the biosensor is formed due to non-covalent interactions into symmetrical oligomers showing increased stability and it is immobilised on a suitable polymer-carrier such as a glutaraldehyde cross-linked chitosan carrier.

The members of Cucurbit[n]uril (CB [n], wherein n=5 to 8) family entail high binding affinity and molecular recognition in aqueous environments, high structural integrity, thermal stability, low toxicity and controlled binding kinetics; thus, they can be utilized in a wide range of conditions for nanomaterial production (Germain et al., 1998; Lagona et al., 2005; Barrow et al., 2016; Cicolani et al., 2021). CB [n] s are symmetric molecules with hydrophobic cavities that are accessed through carbonyl rims (Assaf & Nau, 2015), and consist of n glycolurils and 2n methylene groups (Cong et al., 2016). The CB [n] homologues are usually able to encapsulate a variety of positively charged guest-molecules in their cavities mostly due to ion-dipole interactions (Mock, 1995; Kim, 2002; Kim et al., 2000; Lagona et al., 2005; Assaf & Nau, 2015). In addition, interactions of CB [n] s with a wide range of metal ions have led to the development of the rapidly growing field of supramolecular chemistry (Ni et al., 2015; Ni et al., 2014). The synthetic macrocyclic host CB[8] consists of eight glycoluril groups that form a hydrophobic cavity which is able to bind one or two guest-molecules, resulting in the formation of homo- or heteromeric complexes. The potential guest molecules of CB[8] usually contain an aromatic group that attaches to the hydrophobic cavity, for instance the side chain of phenylalanine at a protein's N-terminus, thus releasing high-energy water molecules, as well as a positively charged side group that forms ion-dipole bonds with its carbonyl groups (Huang et al., 2016; Smith et al., 2015; van Dun et al., 2017). CB[8] is able to simultaneously bind two aromatic side chains of the N-terminal tripeptides Trp-Gly-Gly (WGG) and Phe-Gly-Gly (FGG) with high binding affinity (Heitmann et al. (2006)). Therefore, construction of supramolecular protein complexes utilise CB[8] as a “molecular glue” based on host-guest interactions with high affinity and selectivity for the N-terminal FGG tag (Biedermann et al., 2011; Hou et al., 2013; Bosmans et al., 2016) or by incorporating the FGG epitope on the outer surface of a protein (Li et al., 2017). The morphology of the resulting complex depends on the location of the recognition site in the protein building block as well as the overall protein structure.

More particularly, in the presence of cucurbit[8]uril, a homo-hexameric structure is formed, adopting an antibody-like (IgG-like) 3D (three dimensional) structure. Such formulated enzyme assemblies (homohexamer) retain their catalytic and binding properties, without significant losses in their catalytic properties. Moreover, such a homohexamer exhibits exceptional improvement in its thermal stability (Tₘ at approximately 85° C. (15° C. increase compared to the dimeric enzyme).

These hexameric protein scaffolds based on GSTs can be an ideal fold for the generation of compounds with a wide spectrum of applications in biotechnology. The structural features of GSTs scaffold, provide several advantages for designing engineered enzyme variants with new catalytic or binding properties. These advantages can be briefly summarized as follows:
a) GSTs display highly divergent functions/specificities and binding/catalytic promiscuity towards a variety of chemical reactions and numerous alternative substrates;
b) GSTs have conserved modular architecture with compact fold, well-organized domains and substrate and ligand binding pockets (G-site, H-site and L-site);
c) recombinant GSTs can be efficiently expressed in high yields in Escherichia coli and purified in a single chromatographic step by affinity methods;
d) numerous crystal structures and complexes (ligand free or ligand bound) and have been determined with high resolution by x-ray crystallography;
e) GSTs exhibit good structural stability with Tₘ values ranging between 50 to 65° C.;
f) GSTs are amenable for engineering by incorporating unnatural amino acids or by chemical conjugation.

This unique structure of the homo-hexameric GST based assemblies of the current invention allows for flexible and/or tunable xenobiotic-binding properties suitable for biosensing, bioscavenging, biocatalysis and drug delivery.

Therefore, according to one aspect the present invention relates to a biosensor comprising a glutathione-S-transferase homohexamer, wherein each transferase monomer of said homohexamer comprises a Phenylalanine-Glycine-Glycine tag at its N-terminal site, wherein said N-tagged monomer is assembled to an homohexamer through binding of Cucurbit[n]uril to said Phenylalanine-Glycine-Glycine tag and wherein said homohexamer is further characterised that it is immobilised on a polymer carrier.

In one embodiment said biosensor is characterized in that said N-tagged monomer is assembled to a homohexamer through binding of Cucurbit[8]uril to said Phenylalanine-Glycine-Glycine tag and the concentration ratio of said N-tagged monomer to Cucurbit[8]uril is 1:1.

In one embodiment said glutathione S-transferase monomer of said biosensor comprises the SEQ ID NO: 1 or SEQ ID NO: 2.

In one embodiment said homohexamer of said biosensor is immobilized on a glutaraldehyde-crosslinked chitosan carrier.

In another aspect the invention relates to a method of production of a biosensor of the invention characterized in that said method comprises the following steps of
mixing a chitosan gel with a glutaraldehyde solution so that a cross-linked chitosan carrier is formed
adding a solution of said glutathione-S-transferase homohexamer to said cross-linked chitosan carrier.

In one embodiment said method further comprises the following steps of
dissolving 2%-4% (w/v) of chitosan in 2% (v/v) acetic acid so that a chitosan gel is formed
storing said chitosan gel for 12 to 24 hours at 4° C.
adding a glutaraldehyde solution at a concentration of 0.4%-1% (v/v), said glutaraldehyde being diluted in 20 mM KH₂PO₄, PH 7.0
washing with 2 mL 20 mM KH₂PO₄, PH 7
storing the cross-linked chitosan carrier for 3 to 10 days
adding a solution of glutathione S-transferase homohexamer at a concentration of 0.5 to 1 mg/mL
incubating said solution added to the carrier for 12 to 24 hours at around 25° C. with stirring at about 50-80 rpm
removing the non-absorbed solution of glutathione S-transferase homohexamer
incubating the carrier with a solution of 2 mg/mL NaBH₄ for about 30 min
washing with 20 mM KH₂PO₄, PH 7 in order to remove the unbound enzyme

In one embodiment said method further comprises the following steps of
dissolving 2%-4% (w/v) of chitosan in 2% (v/v) acetic acid so that a chitosan gel is formed
storing said chitosan gel for 12 to 24 hours at 4° C.
adding a glutaraldehyde solution at a concentration of 0.4%-1% (v/v), said glutaraldehyde being diluted in 20 mM KH₂PO₄, PH 7.0
washing with 2 mL 20 mM KH₂PO₄, PH 7
storing the cross-linked chitosan carrier for 3 to 10 days
adding a solution of glutathione S-transferase homohexamer at a concentration of 0.5 to 1 mg/mL
incubating said solution added to the carrier for 12 to 24 hours at around 25° C. with stirring at about 50-80 rpm
removing the non-absorbed solution of glutathione S-transferase homohexamer
incubating the carrier with a solution of 2 mg/mL NaBH₄ for about 30 min
washing with 20 mM KH₂PO₄, PH 7 in order to remove the unbound enzyme

In a further aspect the invention relates to a method of detecting a xenobiotic in a sample using a biosensor of the present invention

In one embodiment said xenobiotic of said method is a herbicide, preferably a chloroacetanilide herbicide, more preferably butachlor and said sample is a water sample.

In one other aspect, the invention relates to a use of the biosensor of the invention for the detection of a xenobiotic, preferably a chloroacetanilide herbicide, more preferably butachlor, in a sample, preferably a water sample.

In an even further aspect, the invention relates to a kit comprising a biosensor of the invention for the detection of a xenobiotic, preferably a chloroacetanilide herbicide, more preferably butachlor, in a sample, preferably a water sample.

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition.

The term “about” or “approximately” means the mentioned value+/−10%, for example about 10 shall mean 9 to 11.

Unless otherwise required by context, singular terms shall include pluralities and plural terms shall include the singular. The use of “or” means “and/or” unless stated otherwise. The use of the term “including,” as well as other forms, such as “includes” and “included,” is not limiting.

The term xenobiotic is used to describe chemical substances that are foreign to animal life, such as plant constituents, drugs, pesticides, cosmetics, flavorings, fragrances, food additives, industrial chemicals and environmental pollutants.

The terms herbicide and pesticide, in the context of the present disclosure, are used interchangeably. The term chtosan refers to a linear polysaccharide composed of randomly distributed β-(1→4)-linked D-glucosamine (deacetylated unit) and N-acetyl-D-glucosamine (acetylated unit). It is made by treating the chitin shells of shrimp and other crustaceans with an alkaline substance, such as sodium hydroxide (CAS Registry Number: 9012-76-4).

The term oligomer in the context of the current disclosure is a molecule that consists of a few repeating units which could be derived, actually or conceptually, from smaller molecules, monomers and can be a dimer, trimer, tetramer, pentamer or hexamer, more preferably a hexamer. The term homoexamer in the context of the current disclosure is a molecule that consist of six identical monomers

While the present invention has been described with reference to the specific embodiments thereof, it should be understood by those skilled in the art that various changes may be made and equivalents may be substituted without departing from the true spirit and scope of the invention using this disclosure as a guide. Having now described certain embodiments in detail, the same will be more clearly understood by reference to the following examples, which are included for purposes of illustration only and is not intended to be limiting.

### EXAMPLES

### Materials

Enzyme substrates, analytical grade salts, reagents, as well as the herbicide butachlor, were purchased from Sigma-Aldrich (USA). Molecular biology reagents (T4 DNA Ligase, High fidelity DNA polymerase) were obtained by Takara Bio (USA). DPN1 restriction enzyme was obtained from Invitrogen (USA) and the mini prep plasmid isolation kit from Macherey-Nagel (Germany). Phi class cytosolic glutathione S-transferase (GSTF) mutants, sh101 (SEQ ID NO: 1) and sh155 (SEQ ID NO: 1), have been constructed by DNA shuffling, as described previously, and were provided by the Enzyme Technology Laboratory of Agricultural University of Athens (Ioannou et al.; Int J Mol Sci; 2022 5; 23 (13): 7469)

### Example 1: Production of a GSTF Self-Assembly Nanostructure in the Presence of cucurbit[8]uril (CB[8])

### Addition of Phenylalanine-Glycine-Glycine (FGG-) to the N-Terminal Site of GSTFs (FGG-GSTFs)

The phenylalanine-glycine-glycine (FGG-) tag was added to the N-terminal site of selected modified GSTFs sh101 (SEQ ID NO: 1) and sh155 (SEQ ID NO: 2), derived by DNA shuffling. This was accomplished by ligation of plasmid PCR products that contained the gene of interest and were enhanced by phosphorylated oligonucleotide primers that included the N-terminal FGG tag (5′ TTT GGA GGA 3′, SEQ ID NO: 5). The primers were designed in order to amplify the entire vector with the addition of FGG codons to the N-terminal site of the GSTF gene. Both studied enzyme forms were in pETite C-His vectors and had identical N-terminal site sequences, therefore the sequence of the phosphorylated forward primer was 5′ ATG TTT GGA GGA GCA CCT GTA AAA GTC TTT GG 3′ (SEQ ID NO: 3) and the reverse primer was 5′ATG TAT ATC TCC TTC TTA TAG TTA AAC 3′ (SEQ ID NO: 4). The PCR was performed at 25 μL final volume by using 12.5 μL CloneAmp™ HiFi PCR Premix by Takara, 10 pmol of forward and reverse primers and approximately 15-30 ng of plasmid DNA as a template. Initial denaturation was carried at 98° C. for 4 min. Afterwards, 35 cycles of denaturation at 98° C. for 10 s, annealing at 60° C. for 15 s and extension at 72° C. for 15 s were performed. The final extension was carried at 72° C. for 10 min. The reaction products were evaluated by 1% (w/v) agarose gel electrophoresis.

Plasmid DNA PCR products were afterwards circulated by T4 DNA ligase, since phosphorylated primers were used in the initial reaction. Ligation was carried by addition of 1 μL 10× ligase reaction solution, 2 μL of PCR product, 6 μL of sterile ddH₂O and 1 μL of T4 DNA ligase by Takara (350 U/μL). The reaction was incubated at 16° C. for 16 hours and then the enzyme was inactivated by incubation at 65° C. for 15 min.

The restriction enzyme DPN1 was used in order to remove the parent plasmid DNA by addition of 1.5 μL 10× buffer and 0.5 μL DPN1 by Invitrogen (10 U/μL) to 10 μL of the ligase reaction products. Sterile ddH₂O was added up to 15 UL final volume. The reaction was incubated for 3½ hours at 37° C. and inactivation of the enzyme was carried at 80° C. for 20 min. The final product was utilized for 5 transformation of DH5α cells and the addition of the FGG epitope was confirmed by DNA sequencing.

### Expression Systems and Purification Methods of FGG-GSTFs

The plasmid containing FGGsh101 was transformed into Rosetta™ 2 (DE3) pLysS cells, whereas the FGGsh155 vector into BL21 (DE3) pLysS cells. E. coli strains were incubated for 20-24 h in 0.2% w/v lactose medium (Georgakis et al., 2020) with 30 μg/mL kanamycin along with 34 μg/mL chloramphenicol only for the Rosetta™ 2 (DE3) pLysS strain. The cells were harvested by centrifugation at 8,000 rpm for 10 min. Enzyme purification was conducted by Ni-IDA-Sepharose affinity chromatography as described in previous publications (Georgakis et al., 2020; Labrou et al., 2001) and the enzyme purity was evaluated by SDS-PAGE.

### Preparation of FGG-GSTF Samples and Addition of CB[8]

The formation of supramolecular protein structures due to host-guest interactions is heavily influenced by the reaction conditions such as protein concentration, pH and ionic strength (Dong et al., 2014; Sun et al., 2016; Si et al., 2016). In this work, the 2:1 and 1:1 concentration ratios of FGG-GSTF to CB[8] were analyzed, due to the previously established high binding affinity between CB[8] and FGG tag (Heitmann et al., 2006). CB[8] stock solution (195 μM) was prepared in 0.1 M HCl by sonication in a water bath for at least two hours. Alternatively, CB[8] can be dissolved in 10 mM PB buffer, pH 7.4, at 50-70 μM final concentration. Dissolution of CB[8] may be assisted by stirring overnight at 280 rpm and 60° C.

Addition of CB[8] to enzyme solution of known concentration was tested in 1:1 and 2:1 stoichiometry, i.e. 15 μM of CB[8] were added to 15 μM of enzyme and respectively 7.5 μM of CB[8] were added to 15 μM of enzyme. Before the addition of the macrocyclic host, the purified GSTF was dialyzed overnight in 20 mM KH₂PO₄, pH 7.0, at 4° C. A “control” reaction was used in order to evaluate the interaction, where appropriate volume of CB[8]'s solvent was added to the enzyme. This is of great significance, especially when the used solvent was an HCl solution, which can significantly affect the pH of the reaction. The host-guest interactions were carried in 50 mM KH₂PO₄, pH 7.14, in order to maintain the structural integrity and stability of the enzyme and so that the addition of 0.1 M HCl wouldn't result at a reduction of pH at a value less than 6.5.

### Example 2: Enzyme Activity and Steady-State Kinetic Analysis of Modified FGG-GSTFs

GST activity assays were carried by using the 1-chloro-2,4-dinitrobenzene (CDNB)/glutathione (GSH) substrate system as described previously (Axarli et al., 2017; Georgakis et al., 2020). Determination of enzyme activity was conducted using 1 mM CDNB and 2.5 mM GSH final concentration in 0.1 M KH₂PO₄, PH 6.5 and 37° C. Kinetic analysis of the potential supramolecular protein complexes was performed as previously described (Axarli et al., 2016, Georgakis et al., 2020) five days after addition of CB[8] to the enzyme solution in 1:1 stoichiometry. Protein precipitates were removed by centrifugation at 10,000 rpm for 10 min prior to the analysis. The steady state data were processed using GraphPad Prism version 7 (GraphPad Prism Software, Inc.). Turnover numbers were evaluated for one active site per subunit of enzyme. Specific activity (SA) was estimated in micromoles per minute per milligram of protein (μmol min-1 mg-1). Determination of protein concentration was carried using the Bradford assay with bovine serum albumin as standard (Bradford, 1976).

The kinetic parameters of FGGsh101 and FGGsh155 were analyzed both in absence of CB[8] and five days after the addition of the macrocyclic host in 1:1 ratio. This was determined according to the fluorescence intensity monitoring experiments, where the reduction of fluorescence emission appeared to reach a plateau after 4-5 days. (see Example 3)

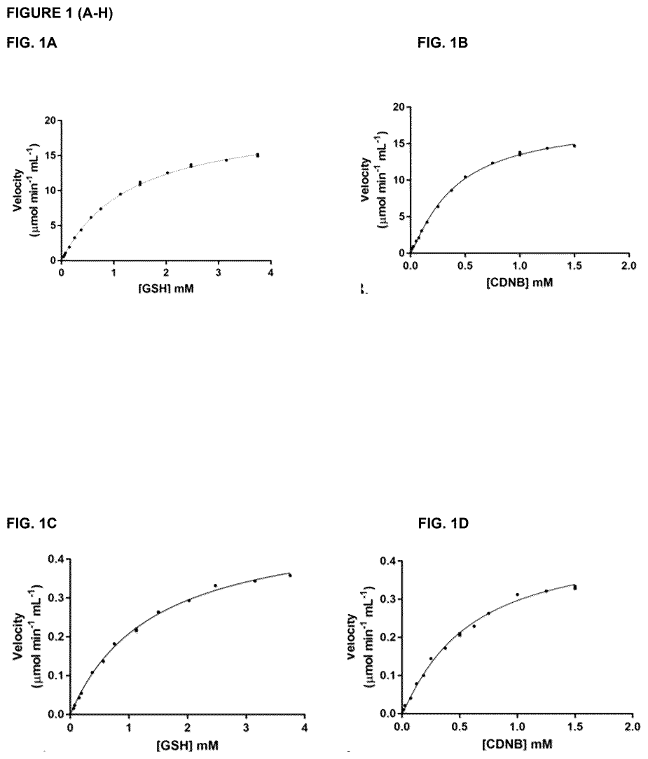

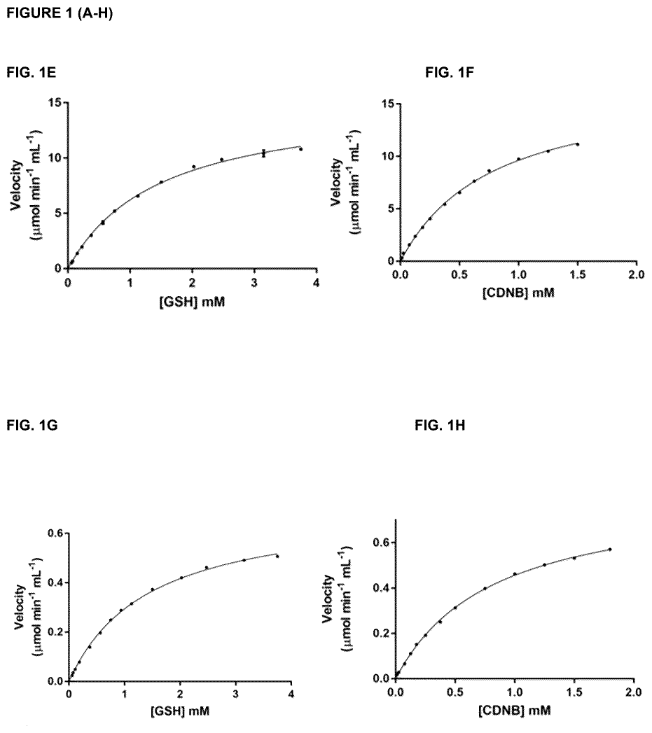

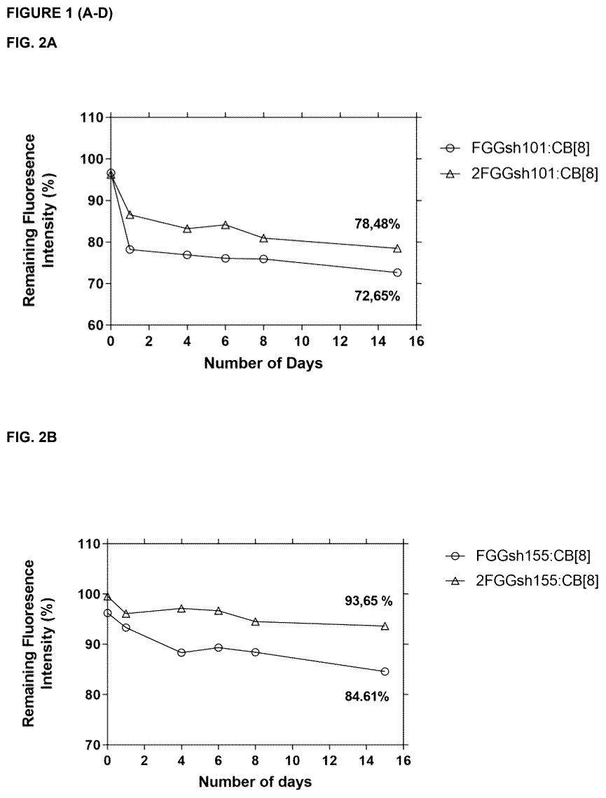

Kinetic analysis did not detect significant differences in the Kₘ and S_{0.5} parameters of the enzyme forms, therefore the host-guest interactions did not result into structural changes that affected their affinity towards the GSH and CDNB substrates (Table 1, FIG. 1). However, their catalytic constants were decreased from the unmodified FGG-GSTF mutants (FGGsh101: 159.4±1.21 min⁻¹, FGGsh155: 168.4±2.72 min⁻¹) to the enzyme samples with CB[8] in 1:1 stoichiometry (FGGsh101: 134.9±3.28 min⁻¹, FGGsh155: 121±1.14 min⁻¹). The decrease in the catalytic constant led to a more significant reduction of their catalytic efficiency (k_{cat}/Kₘ) for the CDNB substrate, which decreased from 397.51 to 240.89 min⁻¹ mM⁻¹ for FGGsh101 and from 218.7 to 149.38 min⁻¹ mM⁻¹ for FGGsh155. However, these values are still comparable to those of the most catalytically potent parent GSTFs of Triticum aestivum and Hordeum vulgare, that were used for the formation of the studied mutants via DNA Shuffling, and have been previously studied (Georgakis et al., 2020).

TABLE 1

| | | | | | k_{cat}/Kₘ | k_{cat}/S_{0.5} |
|---|---|---|---|---|---|---|
| | Kₘ | S_{0.5} | | | (min⁻¹ | (min⁻¹ |
| | (mM) | (mM) | | k_{cat} | mM⁻¹) | mM⁻¹) |
| | GSH | CDNB | n_{H} | (min⁻¹) | GSH | CDNB |
| FGGsh101 | 1.36 ± 0.02 | 0.4 ± 0.02 | 1.17 ± 0.03 | 159.4 ± 1.21 | 118.68 | 397.51 |
| FGGsh101:CB[8] | 1.41 ± 0.05 | 0.56 ± 0.06 | 1.1 ± 0.06 | 134.9 ± 3.28 | 95.67 | 240.89 |
| FGGsh155 | 1.48 ± 0.03 | 0.77 ± 0.07 | 1.01 ± 0.03 | 168.4 ± 2.72 | 113.78 | 218.7 |
| FGGsh155:CB[8] | 1.43 ± 0.03 | 0.81 ± 0.05 | 1.01 ± 0.02 | 121 ± 1.14 | 84.62 | 149.38 |

### Example 3: Fluorescein Isothiocyanate (FITC) Labeling of FGG-GSTFs and Fluorescence Monitoring Experiments

FITC labelling of FGG-GSTFs of example 2, both in presence and absence of CB[8], was carried based on previously published methods (Hermanson, 2013; Ioannou et al, 2022). In brief, selective labeling of FITC on the enzymes' lysines was carried in 0.1 M Sodium carbonate buffer, pH 9, and on cysteines in 0.1M KH₂PO₄, pH 6.5. After addition of CB[8] to the FITC-enzyme at different molar ratios with 1:1 and 2:1 stoichiometry (15 μM CB[8] to 15 μM FITC-FGG-GSTF, 7.5 μM CB[8] to 15 HM FITC-FGG-GSTF), fluorescence quenching phenomena were evaluated by monitoring fluorescence intensity of a standard sample volume (5 μL) in 2 mL of 20 mM KH₂PO₄, PH 7.0, and 25° C., over the course of a few days. Fluorescence emission was monitored in the range of 500 nm to 600 nm using 470 nm excitation wavelength. A sample of FITC-labeled enzyme with an appropriate amount of CB[8] solvent (0.1 M HCl) was used as a “control” reaction. 100% fluorescence intensity corresponds to the labeled enzyme in absence of CB[8]. Moreover, FITC-labeled enzymes were evaluated by confocal laser scanning microscopy (CLSM). The protein precipitate and supernatant were separated on day 9 (lysine FITC labeling) and 7 (cysteine FITC labeling) after addition of CB[8], by centrifugation at 10,000 rpm for 10 min. The separated samples were observed with Axio Observer Ze1/7 microscope (laser wavelength: 488 nm 2.3%, pinhole 100 μm), using suitable filters for FITC in the range of 400-565 nm. The precipitates were re-dissolved in 200 μL of 50 mM KH₂PO₄, pH 7.14, before observation with CLSM. There wasn't visible precipitated protein in the sample 2 FGGsh155:CB[8], thus the observation was limited to the supernatant of the sample.

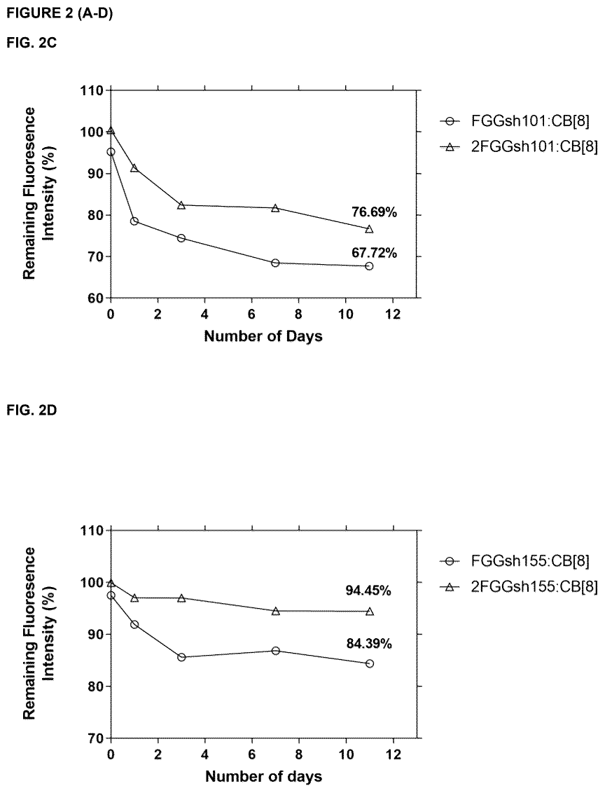

Monitoring of fluorescence intensity of the FITC enzyme solutions indicated a gradual reduction in emission after addition of CB[8], which is possibly associated with the formation of oligomers due to host-guest interactions (Li et al., 2017), as well as the formation of protein precipitate which was not detected in the “control” reactions. Determination of the gradual reduction in fluorescence intensity of the labelled enzyme solution was measured for several days. Both enzymes exhibited a more pronounced reduction in fluorescence intensity in 1:1 enzyme to CB[8] stoichiometry, regardless of the labeling conditions. FIG. 2, which depicts the residual fluorescence intensity, show a sharp reduction in emission on the first day after addition of the macrocyclic compound, followed by a more gradual reduction which is relatively stable after the fourth day from CB[8] addition.

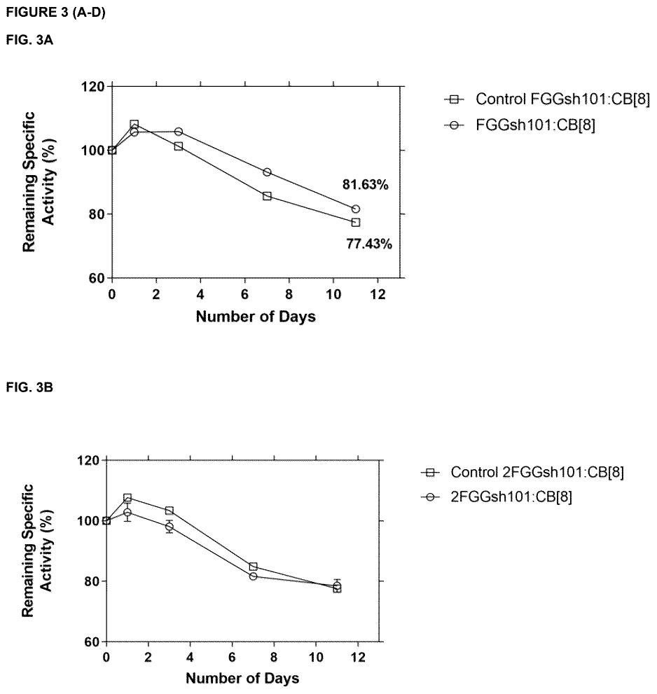

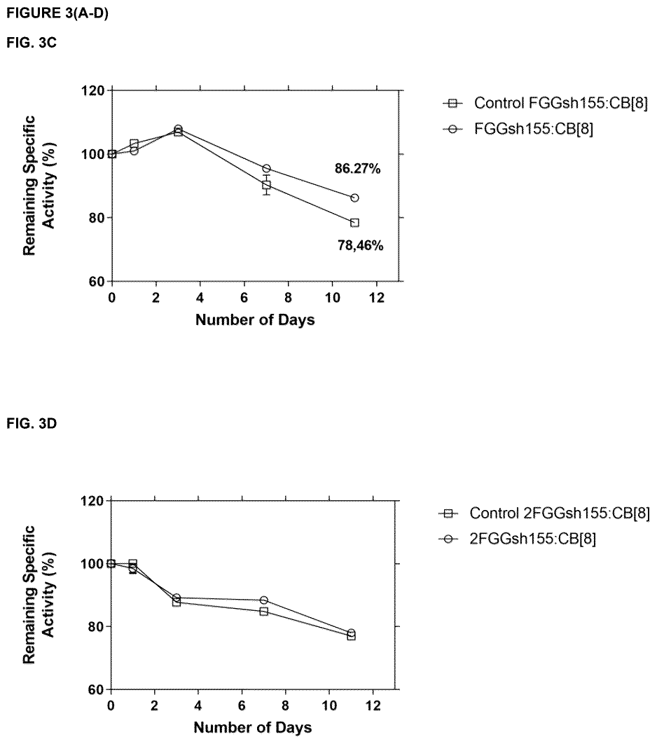

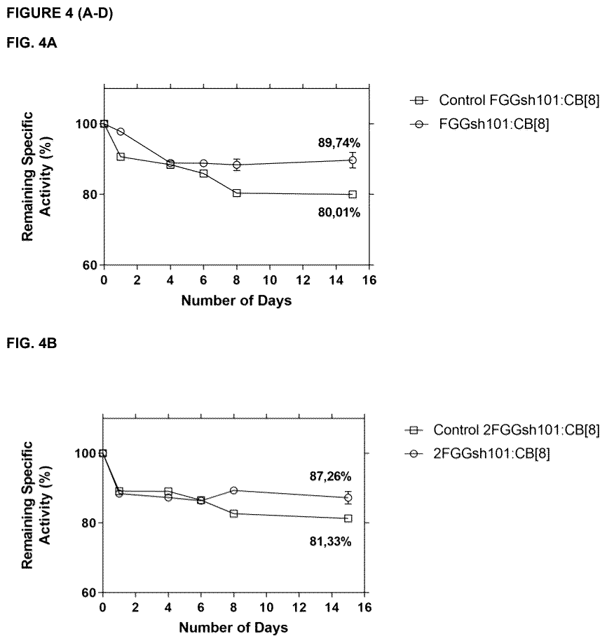

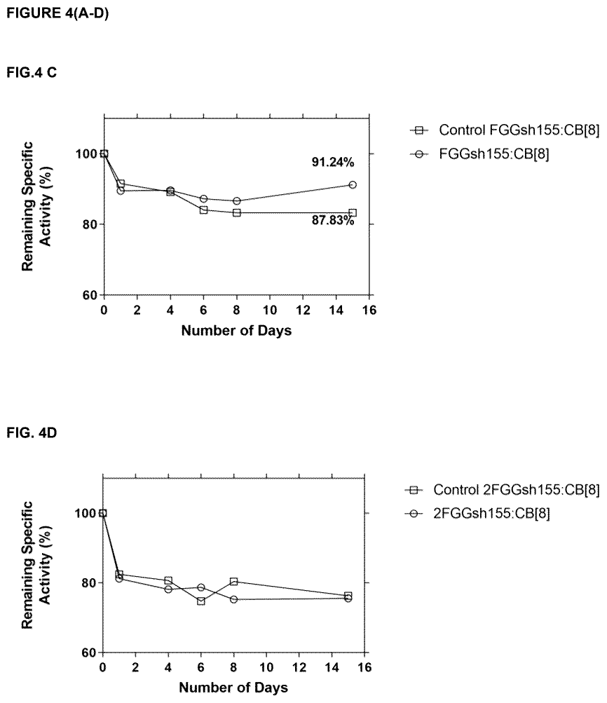

Specific activity of FITC labelled enzymes was determined using the CDNB/GSH substrate system over the course of a few days. Similar results were established for both labeling strategies depending of the pH of the reaction. Specific activity of FITC labelled enzyme samples seems to be slightly better preserved in the presence of CB[8] in 1:1 stoichiometry for both FGGsh101 and FGGsh155 compared to the “control” reaction. Remaining specific activity after almost two weeks of measurements is similar to the “control” reaction in the 2:1 ratio (FIGS. 3 & 4). The supernatant and precipitate of FITC-FGG-GSTFs were separated approximately one week after the addition of CB[8] for observation by confocal laser scanning microscopy (CLSM), see Example 4. After separation, GST activity was detected in the supernatant and the redissolved precipitate; however it was comparatively reduced in the latter (Table 2).

TABLE 2

| | | Specific Activity (U/mg) | |
|---|---|---|---|
| FITC labeling pH | | 9.0 | 6.5 |
| FGGsh101:CB[8] | “Control” | 1.952 ± 0.009 | 2.254 ± 0.02 |
| (1:1) | Supernatant | 1.756 ± 0.027 | 1.999 ± 0.026 |
| | Precipitate | 0.818 ± 0.04 | 0.932 ± 0.009 |
| 2 FGGsh101:CB[8] | “Control” | 1.924 ± 0.009 | 2.332 ± 0.009 |
| (2:1) | Supernatant | 1.910 ± 0.012 | 2.29 ± 0.005 |
| | Precipitate | 0.887 ± 0.002 | 0.959 ± 0.014 |
| FGGsh155:CB[8] | “Control” | 1.641 ± 0.018 | 1.778 ± 0.008 |
| (1:1) | Supernatant | 1.460 ± 0.012 | 1.535 ± 0.019 |
| | Precipitate | 0.407 ± 0.027 | 0.450 ± 0.016 |
| 2 FGGsh155:CB[8] | “Control” | 1.619 ± 0.009 | 1.799 ± 0.008 |
| (2:1) | Supernatant | 1.492 ± 0.01 | 1.588 ± 0.035 |
| | Precipitate | 0.477 ± 0.017 | N/A |

### Example 4: Morphology Study of FITC Labelled Modified GSTF Enzymes after Addition of CB[8] by Confocal Laser Scanning Microscopy (CLSM)

The morphology of supernatant and precipitate samples, as described in Example 3, of FITC labelled enzymes after addition of CB[8] was studied by CLSM.

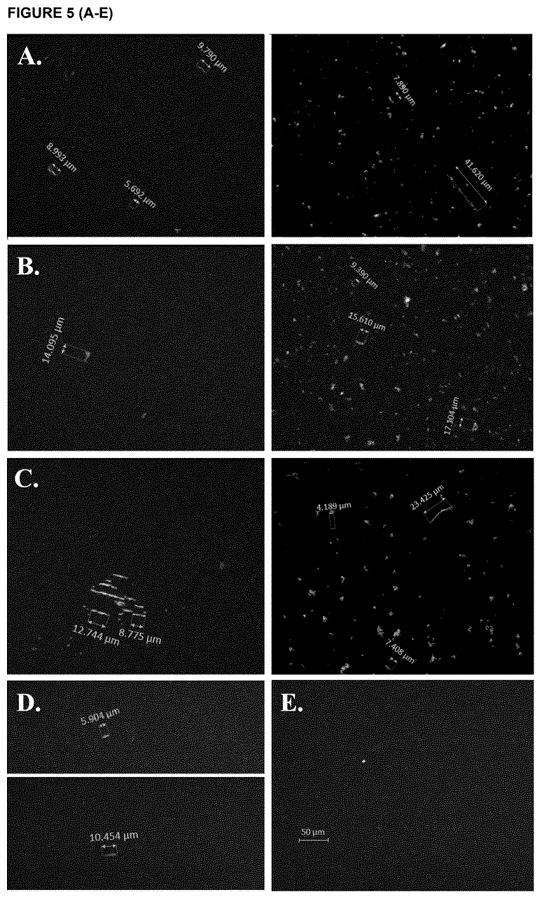

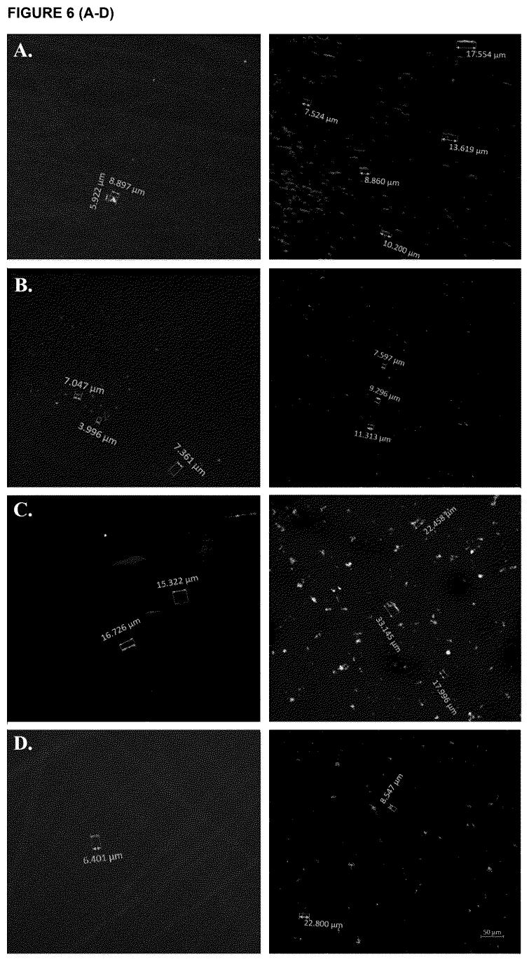

Examination of CLMS images showed obvious differences between the “control” reaction in absence of CB[8] and the supernatant/precipitate of the samples containing CB[8] at 1:1 and 2:1 stoichiometry. The pH of the labelling reaction did not differentiate the morphology of the samples. Control reactions in all conditions exhibited similar sporadic uniformly shaped spots that probably correspond to random protein aggregates (FIG. 5E). However, protein aggregates with irregular or linear shape and length up to dozens of μm were identified in the supernatant samples. Interesting results were also acquired in the formed precipitates, where higher concentrations of linear and irregularly shaped protein aggregates were identified (FIGS. 5 & 6). These formations indicate the potential presence of protein nanostructure complexes due to non-covalent interactions. Since the width of these structures is estimated at approximately 1-1.5 μm and that of the GST dimer is estimated at 4 nm, it is appreciated that these correspond to “superwire” formations, i.e. multiple protein nanostructures bound by non-covalent interactions (Li et al., 2017), thus they become visible by confocal microscopy. In addition, these formations are more likely to precipitate, therefore they are found at higher concentration in the precipitated protein samples.

### Example 5: Determination of Enzymatic Thermal Stability (Tₘ Determination) of FGGsh101 and FGGsh155 in the Presence of the CB[8], Using Differential Scanning Fluorimetry

The thermostability of FGG-GSTFs, FGGsh101 and FGGsh155 was assessed by differential scanning fluorimetry (DSF) in the presence of the macrocyclic compound CB[8]. CB[8] was added in stoichiometry of 1:1 and 2:1 enzyme to host concentration. The reaction was performed in 50 mM KH₂PO₄ solution, pH 7.14, using either 15 μM enzyme and 15 μM CB[8] (1:1) or 15 μM enzyme and 7.5 μM CB[8] (2:1). A “control” reaction for each ratio was also measured, where only CB[8]'s solvent of was added to the enzyme. Differential scanning fluorimetry was performed in 20 mM KH₂PO₄ buffer, pH 7, with 4 μg of protein sample, according to published methods (Huynh & Partch, 2015). Thermal shift assays we carried using a Real-time PCR StepOne™ instrument (Applied Biosystems, USA) and the SYPRO Orange protein dye at a temperature range between 15° C. and 99° C. with a ramping rate of 1%. Temperature of protein denaturation midpoint (Tₘ) was estimated by the first derivative of the normalized fluorescence data.

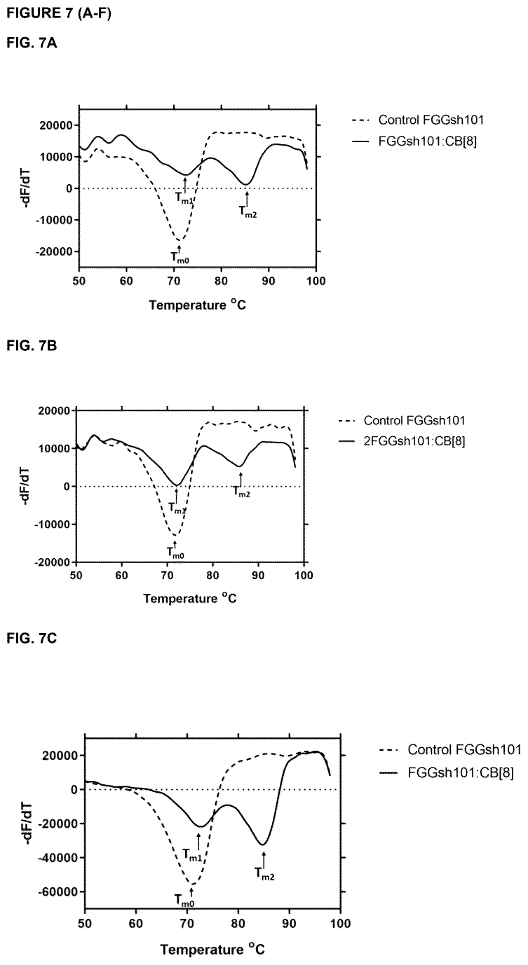

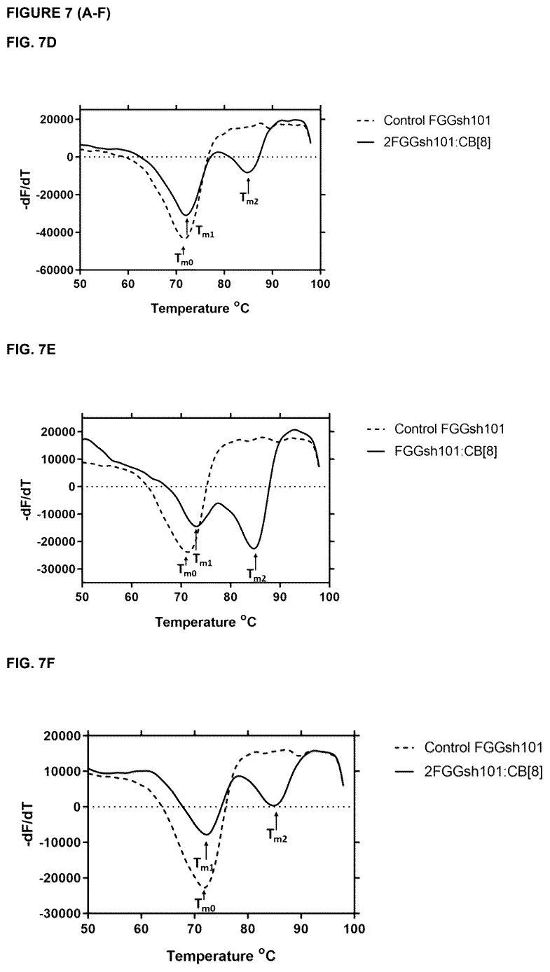

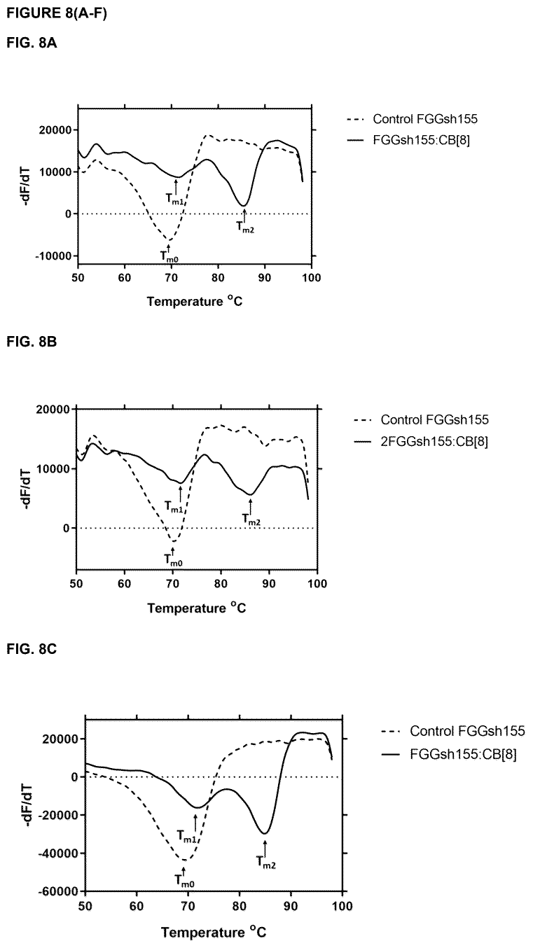

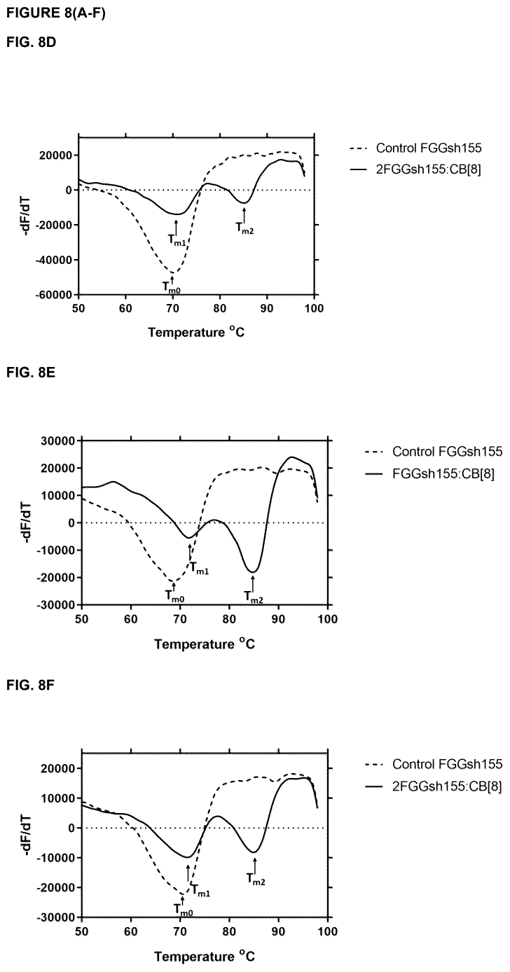

The addition of CB[8] to both FGGsh101 and FGGsh155 resulted in the appearance of a second distinct melting point (Tₘ₂≈85° C.) with 13-15° C. increased value than Tₘ₀ (Table 3 & 4). Tₘ₀ refers to the melting point of the “control” reactions, where CB[8] was absent. Since two prominent Tₘ values were detected after exposure to the macrocyclic compound, it is possible that different populations of enzyme complexes with significantly improved thermostability are formed as a result of host-guest interactions (FIGS. 7 & 8). In addition, an increase in the Tₘ₁ value was found, which is similar and corresponds to the Tₘ₀ value of the control reaction, especially in the 1:1 stoichiometry tests. Approximately 1.5-2° C. increase in Tₘ₁ was evaluated for FGGsh101 and up to 3.5° C. for FGGsh155 (Table 3 & 4). The comparative change of Tₘ₁ and Tₘ₂ was studied for a few days (16), however it was evident from the day of CB[8] addition. The first derivative graphs derived by normalized fluorescence data showed a more pronounced Tₘ₂ peak (approximately 85° C.) than that of Tₘ₁ (approximately 70° C.) in the 1:1 ratio, while the opposite was observed for the 2:1 enzyme concentration to CB[8] (FIGS. 7 & 8). Furthermore, the peaks appear less sharp during the day of addition, which is consistent with a more gradual formation of different enzyme complexes that were not detected in the “control” reaction. Therefore, increasing the concentration of CB[8] is considered to contribute to further stabilization of the binding protein region which is otherwise denatured at lower Tₘ (Senisterra et al., 2012; Gao et al., 2020) and induce the formation of stable enzyme complexes in an uneven population of structures, since in 1:1 stoichiometry samples the first derivative curve of Tₘ₂ is sharper. In parallel with the DSF analysis, the enzyme activity of the samples was assessed for the substrate system CDNB/GSH with similar findings to the FITC labelled enzymes.

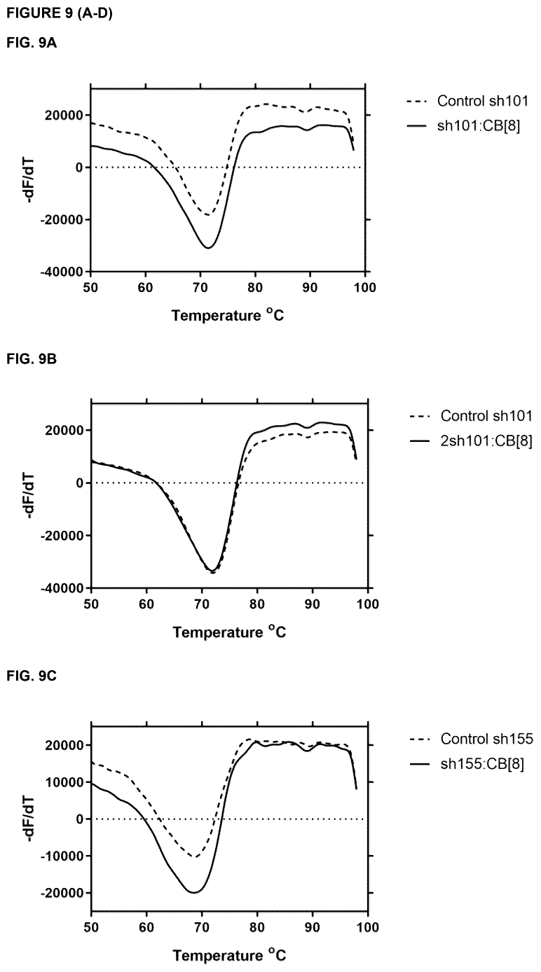

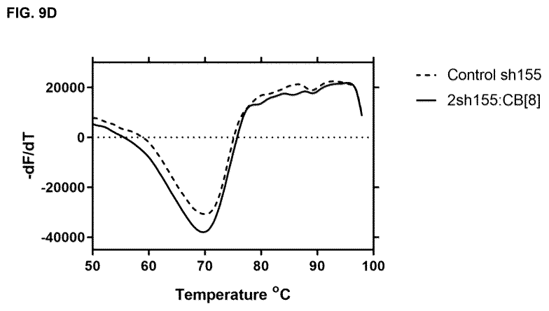

In order to further confirm the effect of the host-guest interactions, thermal shift assays were carried for the enzyme forms sh101 and sh155 that weren't FGG-tagged, in the same conditions as FGGsh101 and FGGsh155. As shown in FIG. 9, addition of CB[8] to sh101 and sh155 enzyme forms did not induce the appearance of an additional Tₘ value or any significant increase of its value. The measurements were carried for almost ten days without any significant changes (Table 5).

TABLE 3

| Number | | | ΔT₁ | | ΔT₂ |
|---|---|---|---|---|---|
| of | | | (Tₘ₁ − | | (Tₘ₂ − |
| Days | Tₘ₀ (° C.) | Tₘ₁ (° C.) | Tₘ₀₎ | Tₘ₂ (° C.) | Tₘ₀) |
| | FGGsh101:CB[8] | | | | |
| 0 | 71.26 ± 0.18 | 72.46 ± 0.17 | 1.2 | 85.26 ± 0.17 | 14 |
| 1 | 70.93 ± 0.17 | 72.69 ± 0.16 | 1.76 | 84.76 ± 0.17 | 13.83 |
| 3 | 70.95 ± 0.17 | 72.12 ± 0.17 | 1.17 | 85.07 ± 0.17 | 14.12 |
| 6 | 70.95 ± 0.16 | 72.66 ± 0.17 | 1.71 | 84.6 ± 0.17 | 13.65 |
| 9 | 71.1 ± 0.17 | 72.57 ± 0.18 | 1.47 | 84.7 ± 0.17 | 13.6 |
| 16 | 71.24 ± 0.17 | 73.02 ± 0.17 | 1.78 | 84.6 ± 0.17 | 13.36 |
| | 2 FGGsh101:CB[8] | | | | |
| 0 | 71.86 ± 0.17 | 72.15 ± 0.18 | 0.29 | 85.56 ± 0.16 | 13.7 |
| 1 | 71.51 ± 0.17 | 71.8 ± 0.17 | 0.29 | 84.47 ± 0.17 | 12.96 |
| 3 | 71.53 ± 0.17 | 72.12 ± 0.17 | 0.59 | 85.36 ± 0.17 | 13.82 |
| 6 | 71.52 ± 0.16 | 71.8 ± 0.16 | 0.28 | 84.6 ± 0.17 | 13.08 |
| 9 | 71.7 ± 0.16 | 71.8 ± 0.26 | 0.1 | 84.7 ± 0.17 | 13 |
| 16 | 71.76 ± 0.09 | 72.12 ± 0.17 | 0.36 | 84.89 ± 0.17 | 13.7 |

* Determined Tₘ values of FGGsh101 after addition of C[8] in 1:1 (FGGsh101:CB[8]) and 2:1 stoichiometry (2 FGGsh101:CB[8]) on selected days. Tₘ₀ corresponds to the melting point temperature of the “control” reaction without CB[8], Tₘ₁ and Tₘ₂ correspond to the peaks that formed after addition of CB[8]. The difference between the means of Tₘ₁ and Tₘ₂ from Tₘ₀ is also included (ΔT₁, ΔT₂).

TABLE 4

| Number | | | ΔT₁ | | ΔT₂ |
|---|---|---|---|---|---|
| of | | | (Tₘ₁ − | | (Tₘ₂ − |
| Days | Tₘ₀ (° C.) | Tₘ₁ (° C.) | Tₘ₀₎ | Tₘ₂ (° C.) | Tₘ₀) |
| | FGGsh155:CB[8] | | | | |
| 0 | 69.45 ± 0.18 | 71.26 ± 0.17 | 1.81 | 85.26 ± 0.17 | 15.81 |
| 1 | 68.86 ± 0.17 | 72.4 ± 0.17 | 3.53 | 85.06 ± 0.17 | 16.19 |
| 3 | 68.59 ± 0.17 | 72.12 ± 0.17 | 3.53 | 85.07 ± 0.17 | 16.48 |
| 6 | 69.25 ± 0.16 | 71.52 ± 0.16 | 2.27 | 84.6 ± 0.17 | 15.35 |
| 9 | 69.35 ± 0.2 | 71.98 ± 0.17 | 2.64 | 85 ± 0.17 | 15.65 |
| 16 | 68.6 ± 0.17 | 71.53 ± 0.17 | 2.93 | 84.6 ± 0.17 | 16 |
| | 2 FGGsh155:CB[8] | | | | |
| 0 | 70.05 ± 0.17 | 71.57 ± 0.17 | 1.52 | 85.85 ± 0.17 | 15.8 |
| 1 | 70.34 ± 0.17 | 70.64 ± 0.17 | 0.29 | 84.18 ± 0.17 | 13.83 |
| 3 | 70.06 ± 0.17 | 70.06 ± 0.17 | 0 | 85.66 ± 0.17 | 15.6 |
| 6 | 70.1 ± 0.16 | 70.95 ± 0.16 | 0.84 | 84.89 ± 0.17 | 14.79 |
| 9 | 70.22 ± 0.17 | 70.51 ± 0.17 | 0.29 | 85 ± 0.17 | 14.78 |
| 16 | 70.65 ± 0.17 | 71.53 ± 0.17 | 0.88 | 84.89 ± 0.17 | 14.24 |

* Determined Tₘ values of FGGsh155 after addition of C[8] in 1:1 (FGGsh155:CB[8]) and 2:1 stoichiometry (2 FGGsh155:CB[8]) on selected days. Tₘ₀ corresponds to the melting point temperature of the “control” reaction without CB[8], Tₘ₁ and Tₘ₂ correspond to the peaks that formed after addition of CB[8]. The difference between the means of Tₘ₁ and Tₘ₂ from T is also included (ΔT₁, ΔT₂).

TABLE 5

| | Control | | Control | |
|---|---|---|---|---|
| | sh101:CB[8] | sh101:CB[8] | 2 sh101:CB[8] | 2 sh101:CB[8] |
| | Tₘ₀ (° C.) | Tₘ (° C.) | Tₘ₀ (° C.) | Tₘ (° C.) |
| Day 0 | 71.51 ± 0.17 | 71.8 ± 0.18 | 71.8 ± 0.18 | 71.8 ± 0.18 |
| Day 1 | 71.25 ± 0.17 | 71.25 ± 0.17 | 71.84 ± 0.17 | 72.13 ± 0.17 |
| Day 3 | 71.32 ± 0.17 | 71.32 ± 0.17 | 71.9 ± 0.16 | 71.9 ± 0.16 |
| Day 9 | 71.56 ± 0.17 | 71.26 ± 0.17 | 71.86 ± 0.17 | 71.86 ± 0.17 |
| | Control | sh155:CB[8] | Control | 2 sh155:CB[8] |
| | Tₘ₀ (° C.) | Tₘ (° C.) | Tₘ₀ (° C.) | Tₘ (° C.) |
| Day 0 | 68.87 ± 0.17 | 68.87 ± 0.17 | 70.04 ± 0.17 | 69.74 ± 0.17 |
| Day 1 | 69.2 ± 0.17 | 69.49 ± 0.17 | 69.9 ± 0.25 | 68.91 ± 0.17 |
| Day 3 | 68.68 ± 0.17 | 68.68 ± 0.17 | 69.85 ± 0.17 | 69.55 ± 0.17 |
| Day 9 | 68.01 ± 0.26 | 67.93 ± 0.09 | 70.19 ± 0.26 | 70.09 ± 0.17 |

* Determined Tₘ values of sh101 and sh155 after addition of C[8] in 1:1 and 2:1 stoichiometry on selected days. Tₘ₀ corresponds to the melting point temperature of the “control” reaction without CB[8].

### Example 6: Assessment of Formation of FGG-GSTs Oligomers with SEC-HPLC

The ability of CB[8] to induce the formation of FGG-GSTs oligomers was studied by SEC HPLC (size exclusion chromatography, high performance liquid chromatography). Enzyme samples of 1:1 and 2:1 stoichiometry of enzyme to CB[8] concentration were analyzed with 1 mL/min flow rate and mobile phase 0.1 M KH₂PO₄, 0.1 M KCl, 0.025% NaN₃, pH 6,5, using the BioSep™ 5 μM Sec-s3000 290 Å column.

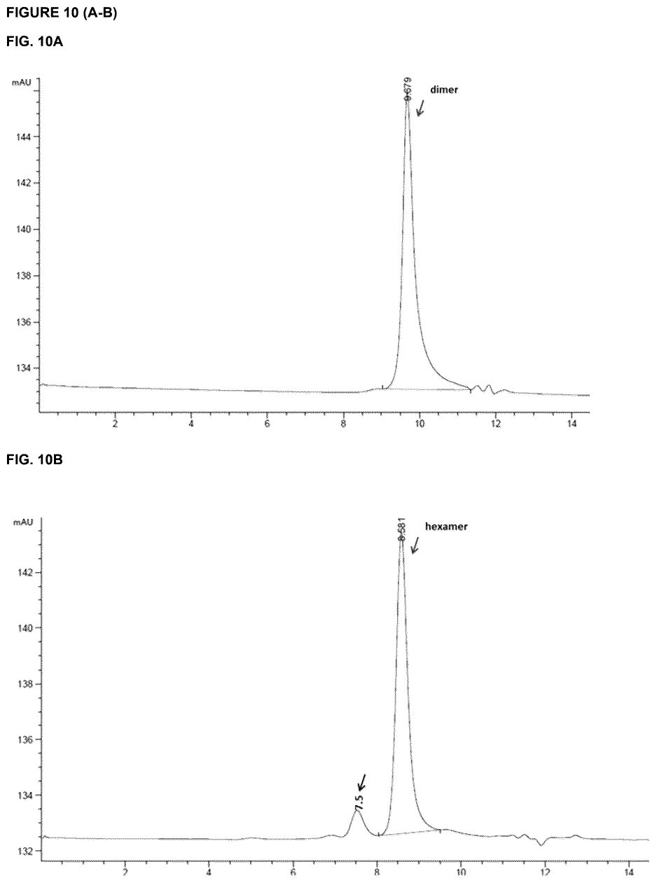

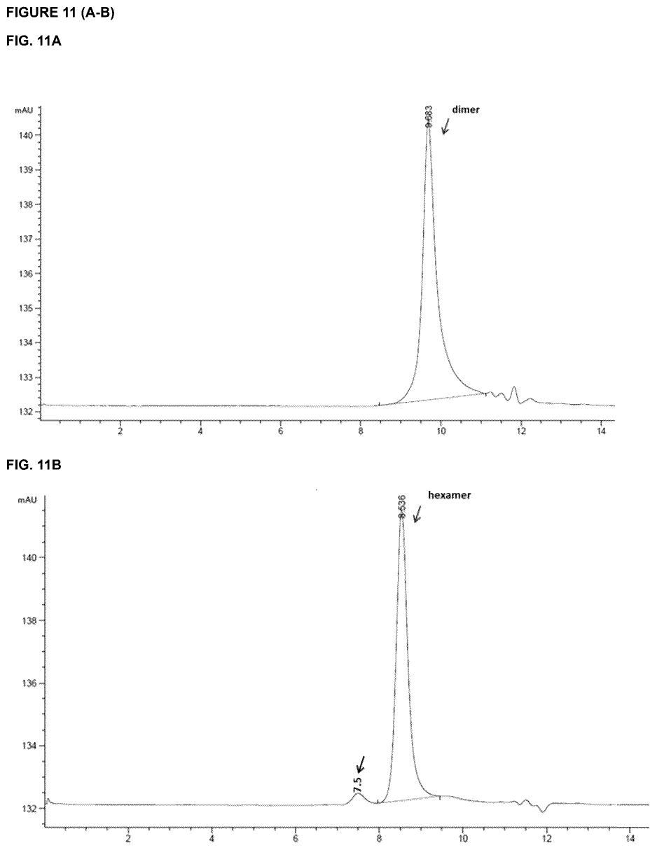

SEC HPLC analysis of 1:1 stoichiometry samples (15 μM of enzyme and 15 μM of CB[8) was carried after completing the DSF analysis and it showed the formation of GST hexamers and possibly even larger polymers induced by CB[8] (FIGS. 10 & 11). In the “control” reactions, a single elution peak was detected at approximately 9.7 min, which corresponds to the molecular weight of GSTF dimers (≈50 kDa). In the 1:1 stoichiometry samples of both FGGsh101 and FGGsh155, an elution peak was found at 8.5 min, which corresponds to hexamer GSTFs (˜150 kDa), as well as a fairly low peak at approximately 7.5 min that corresponds to even larger oligomeric complexes. Almost complete conversion of the dimer to a hexamer complex was detected in the 1:1 stoichiometric samples. On the other hand, concentration ratio 2:1 samples did not exhibit such clear results, since in the 2 FGGsh101:CB[8] sample only the peak of a dimer was detected and in the 2 FGGsh155:CB[8] sample the found peaks corresponded to GST dimers and hexamers though their signal was significantly reduced. Therefore, it is possible that 2:1 stoichiometry enzyme samples did not exhibit enough uniformity and sufficient concentration of GST oligomers to be detected by SEC HPLC under these conditions.

### Example 7: Morphology Assessment of FGG-GSTF Mutant Oligomers with Scanning Electron Microscopy (SEM)

The morphology of GSTF mutant FGGsh155:CB[8] (FGG-GSTF mutant 15 μM and 15 μM CB[8]) was analyzed by scanning electron microscopy (SEM). The sample was prepared by overnight dialysis of purified FGGsh155 in 20 mM KH₂PO₄, PH 7, at 4° C. before the addition of CB[8] in 1:1 ratio. Five days after addition of CB[8], the sample FGGsh155:CB[8] was dialyzed in 1 mM KH₂PO₄, pH 7, in order to remove excess buffer salts. The preparation for SEM involved depositing a small amount of sample on a silicon substrate, drying in air and charring.

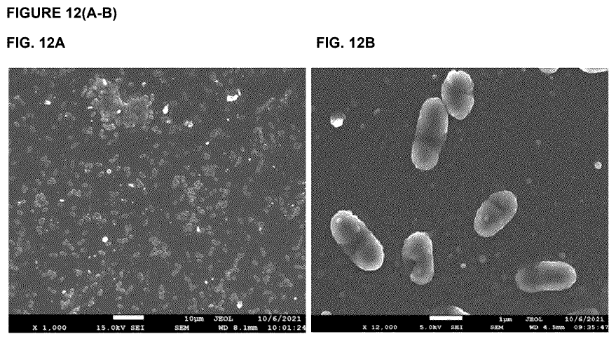

Uniformly arranged particles with a rather cylindrical shape and dimensions up to 1 μm were detected in the “control” sample of FGGsh155, which was prepared by addition of CB[8]'s solvent (0.1 M HCl) to the enzyme. These particles formed agglomerates in some areas and appear thinner in the center either due to a hollow inside or a connection of two individual particles (FIG. 12). However, due to their size they cannot be identified as GSTFs and they did not appear in the sample where CB[8] had been added.

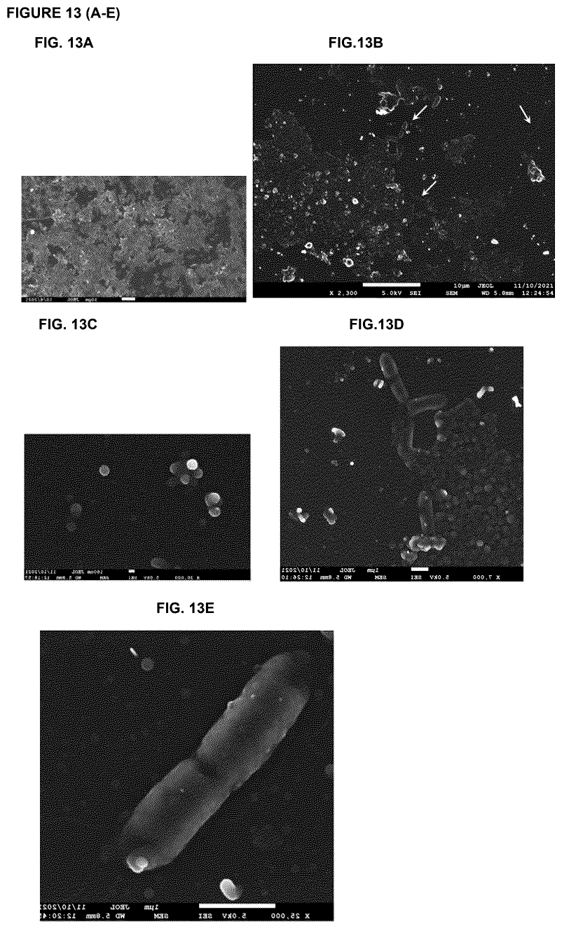

SEM analysis of FGGsh155:CB[8] indicated the formation of a layer of spherical particles measuring 100-200 nm, which covers most of the silicon substrate (FIG. 13A). Further investigation after 1:20 dilution of the sample indicated linear particle complexes with approximately 3-4 μm length and 1 μm width, which very likely correspond to the previously detected linear formations of FITC-labeled enzymes by CLSM (confocal laser scanning microscopy) These formations are considered the outcome of complexation of numerous nanospheres of 100-200 nm into cylindrical or linear formations that scale up to a few μm in length. In addition, a few fine linear structures of a few tens of μm were observed (FIG. 13B), which probably correspond to linear protein nanostructures with consistent morphology to previous publications (Li et al., 2017; Hou et al., 2013). These formations are likely to result from the entry of two N-terminal Phe into the cavity of a CB[8], thus successively adhering dimer GSTF molecules in a linear formation.

### Overall Description of FGGsh155:CB[8] Crystal Structure

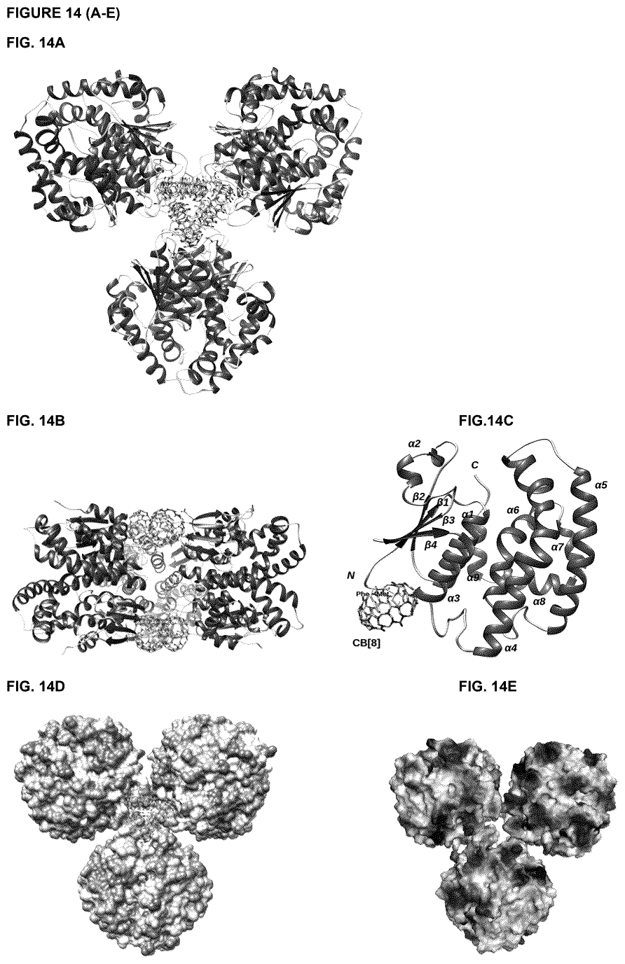

The crystal structure of the FGGsh155:CB[8] revealed the formation of a symmetric GSTF hexamer, where each GST dimer is interacting with two different CB[8] molecules (FIG. 14). Each CB[8] hydrophobic cavity is occupied by the N-terminal methionine and phenylalanine residues of each GSTF subunit. In addition, CB[8] s form two consecutive triangles on either side of the hexamer due to non-covalent interactions. The triangulation of supramolecular host-guest complexes in 1:1 ratio is a rare phenomenon that is not completely understood and has previously been reported in CB[8] with nitroxide or its analogues and sodium cations (Combes et al., 2019; Bardelanget al., 2009; Mileo et al., 2009).

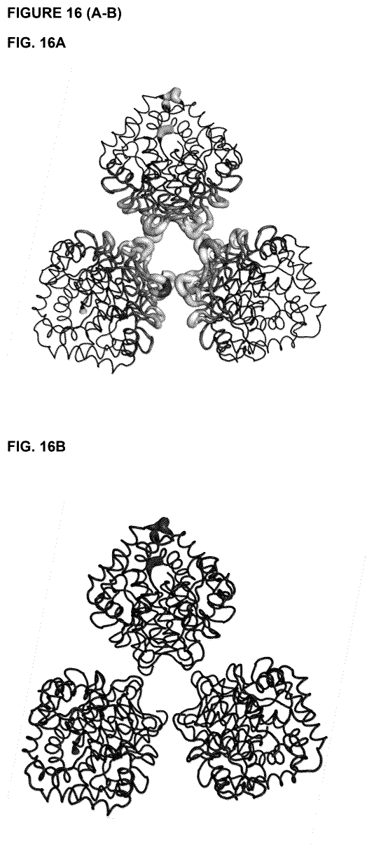

The dimer building blocks of the hexamer GSTF complex exhibited the usual structural profile of cytoplasmic GSTs (Georgakis et al., 2020; 2021; Perperopoulou et al., 2021; Sylvestre-Gonon et al., 2019; Chronopoulou et al., 2018; Axarli et al., 2017), as did the sh155 mutant that did not carry the FGG-epitope and was analyzed previously (loannou et al 2022). Briefly, each homodimer subunit has a smaller N-terminal region with a thioredoxin-like motif (residues 1-78) and a larger C-terminal region (residues 92-213) consisting only of α-helixes. The dimer subunits consist of a β-sheet surface formed by β1 (Val4 to Phe7), β2 (Tyr29 to Val32), antiparallel β3 (Ala57 to Val60) and β4 (Leu63 to Phe66) sheets, that are located among the α-helices, α1 (Thr13 to Val25), α2 (Phe36 to Leu47) and α3 (Ser68 to Lys78). At the N-terminus the amino acid residues Met (−2 numerical position in the amino acid sequence) and Phe (−1 position) are located within the hydrophobic cavity of the macrocyclic host CB[8]. The C-terminal region of each dimer subunit consists of six α-helices: α4: Leu92-Met126, α5: Gln133-Gln156, α6: Phe167-Ala181, α7: Ala185-Ser190, α8: Pro192-Ala 203 and α9: Pro205-Thr213 (FIG. 16C).

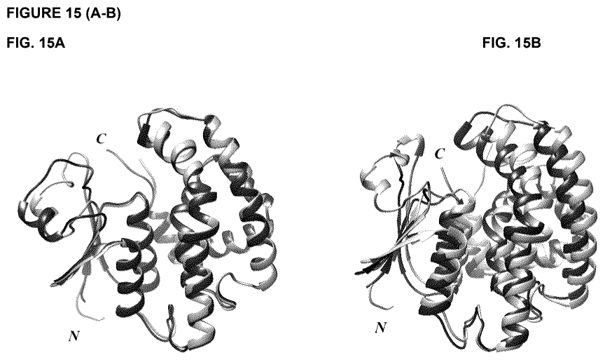

Superimposition of the crystal structure of sh155 (PDB code: 7ZA4) and subunits C and E of the hexamer FGGsh155:CB[8] resulted in a root mean square deviation (RMSD) of 0.477, indicating little structural differences between the molecules, that may be attributed to the non-covalent interactions that resulted to the hexamer formation, since their amino acid sequence is identical (FIG. 15). The deformation energy, which correlates with the local flexibility of a protein molecule, as well as the atomic fluctuation, that estimates the amplitude for the absolute atomic motion, were calculated by normal mode analysis (Rodrigues et al., 2018) (FIG. 16). The area around protein binding site of the CB[8] showed increased protein flexibility in the deformation energy plot, which indicates dynamic differences compared to the sh155 dimer structure (loannou et al., 2022).

Hexamer Formation Due to Non-Covalent Interactions with CB[8]

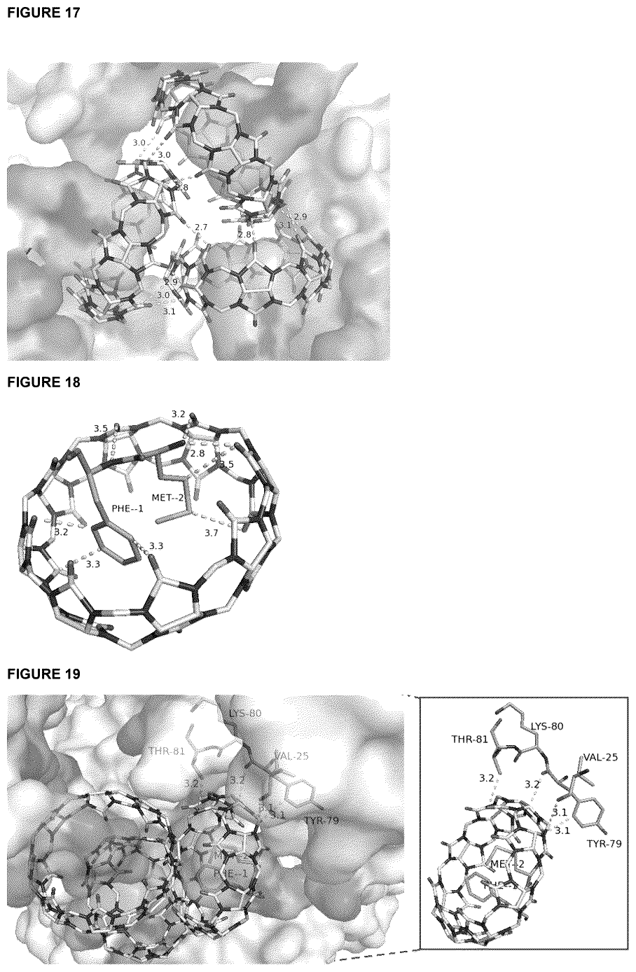

Each GSTF subunit of the hexamer molecule interacts with a CB[8] molecule which in turn interacts with two other macrocyclic hosts, thus forming two successive triangular complexes connecting three GSTF homodimers (FIG. 14). The non-covalent interactions between the formed CB[8] triangles and the enzyme that carries the FGG-tag probably determine the final shape of the hexamer. The outer surface of CB[8] is positively charged (Zhang et al., 2005; Ni et al., 2013), though the carbonyl groups at its rims as well as the inner surface of its cavity are negatively charged (Ni et al., 2014). Based on the estimated distances between the carbonyl rims of one host to the amino or methyl groups of the neighbor's outer surface, this triangular formation occurs due to hydrogen bonds between the macrocyclic hosts (McRee, 1999; Jeffrey, 1997) (FIG. 17). 20

In addition to the formed CB[8] triangles, other non-covalent interactions between CB[8] and the enzyme subunits may have assisted to the hexamer creation. Initially, the phenylalanine side chain of the FGG tag is located into the cavity of the macrocyclic host; however, the second available site in the cavity is covered by the first methionine that precedes Phe in the amino acid sequence, instead of an N-terminal phenylalanine of another subunit. In addition to the Phe and Met side chain interaction with the cavity, there is formation of hydrogen bonds between the N-terminal and protein backbone amino groups with the carbonyl rims of the cavity (FIG. 18). CB[8] is able to bind two N-terminal FGG-tripeptides with high affinity (Heitmann et al., 2006), also the binding of two Phe is possible even when they are at an intermediate position of a peptide chain (Sozini et al., 2013). Since methionine is less hydrophobic than phenylalanine, it is not expected to exhibit strong binding affinity towards CB[8]. The most favorable molecules for selective binding into CB[8]'s cavity usually carry an aromatic group (π-π interactions) or a positively charged side group that enables the formation of ion-dipole interactions with its carbonyl rims (Huang et al., 2016). Therefore, the prevention of a second Phe introduction into the cavity is probably related to the formation of the CB[8] triangle. Furthermore, other non-covalent interactions between the enzyme and CB[8]'s outer surface may have contributed to the final configuration of the hexamer. The residues Lys80, Thr81 and Tyr79, which are located in the sequence that connects the N- and C-terminal domain of the GSTF subunits, were identified at a distance of 3.1-3.2 Å from CB[8]. In addition, some residues of the loop between α1-helix and β2-sheet, i.e. from Val25 to Glu28, may form strong non-covalent bonds with the host molecule (distance of 2.9 to 3 Å) (FIG. 19). However, a different amino acid in each subunit of the hexamer was estimated with possible involvement in the interaction. The amino acids in this region were Val25, Gly26, Ala27 and Glu28.

### Example 8: Covalent Immobilization of FGGsh155:CB[8] on a Chitosan Film by Cross-Linking with Glutaraldehyde (GA)

An enzyme biosensor for the detection of the herbicide butachlor in water samples was developed by immobilisation of FGGsh155:CB[8] on a chitosan carrier by cross-linking with glutaraldehyde (GA) as described previously. (Hung et al., 2003; Jóźwiak et al., 2017; Akakuru & Isiuku, 2017; Khodaei et al., 2018; Vasilieva et al., 2021).

Initially, 2%, 3% and 4% (w/v) medium molecular weight chitosan (molecular weight of 190 to 310 kDa) was dissolved by continuous stirring in 2% (v/v) acetic acid. The formed chitosan gels were stored at 4° C. until the following day (12 to 24 hours) in order to remove any trapped air. For the immobilisation, 180 mg of chitosan gel were deposited by a syringe at the bottom of a cuvette with 4 mL capacity. Then 50 μL of different concentrations of GA solution [0.4%, 0.5%, 0.6%, 0.7%, and 1% (v/v)] were added and the mixture was lightly stirred with a pipette tip. The carrier mixture was left for an hour so that the reaction of GA binding to the amino groups of the carrier was accomplished. GA was diluted in 20 mM KH₂PO₄, PH 7.0. Afterwards, the carrier was washed thrice with 2 mL 20 mM KH₂PO₄, PH 7, in order to remove any unbound GA molecules. The immobilization of FGGsh155:CB[8] was performed five days after the addition of the CB[8] macrocyclic agent at stoichiometry of 1:1, for uniformity of the experiments. Thus, 600 μL of the enzyme solution with 0.7-0.8 mg/mL concentration were added to the activated chitosan medium. The enzyme was incubated with the carrier at 25° C. overnight with stirring at 50-80 rpm. The following day, the volume of the enzyme solution that wasn't absorbed by the carrier was removed and the carrier was incubated with 600 μL of 2 mg/mL NaBH₄ solution for 30 min. This step was conducted in order to stabilize the Schiff's bases that form due to the reaction of GA with the enzyme amino groups (Barbosa et al., 2014). Finally, three washes were performed with 20 mM KH₂PO₄, PH 7, for the removal of any unbound enzyme. Protein binding efficiency to the carrier (%) and retention of enzyme activity (%) were defined as:

$$Protein binding efficiency ( \% ) = \frac{mg of immobilised protein}{mg of protein before immobilisation}$$

$$Retention of enzyme activity ( \% ) = \frac{Specific activity of immobilised enzyme}{Specific activity before immobilisation}$$

Initially, the viscosity of chitosan at of 3 and 4% (w/v) was preferred in order to ensure the stability of the carrier.

### Example 9: Assessment of Enzyme Activity and Kinetic Analysis of the Immobilised Enzyme Complex

Determination of the enzyme activity for the immobilised enzyme complex FGGsh155:CB[8] was performed using the CDNB/GSH substrate system with 20 mM KH₂PO₄, PH 7, as a reaction solution at 37° C., as has been previously described (Georgakis et al., 2020a; Axarli et al., 2017; Cho et al., 2005). The rate assay was recorded for 300 s at 340 nm.

Table 1 shows the enzyme activity for the tested immobilization conditions (see Example 8). Since a significant increase in enzyme activity was determined in 20 mM KH₂PO₄, PH 7, instead of pH 6.5, which is commonly used for GSTFs, the experiments were conducted in pH 7. GST activity generally tends to improve in alkaline conditions with the substrate system CDNB/GSH. The improvement in activity at pH 7.0 was also determined for the enzyme FGGsh155 before immobilization. Comparatively, the optimal enzyme activity was found for 4% (w/v) chitosan carrier, after crosslinking with 50 μL of 0.5% (v/v) glutaraldehyde solution and final incubation for 16 h with 0.8 mg/mL enzyme solution (Table 6). The protein binding efficiency was determined at 34.5% and the retention of enzyme activity was 0.37% in these conditions.

TABLE 6

| Percentage of | | | |
|---|---|---|---|
| chitosan in 2% | Percentage of | Enzyme activity | |
| (v/v) acetic acid | GA solution | (μmol min − 1 mL − 1) | |
| [% (w/v)] | [% (v/v)] | pH 6.5 | pH 7.0 |
| 4 | 0.4 | 0.001 ± 0.0002 | 0.003 ± 0.0001 |
| | 0.5 | 0.002 ± 0.00001 | 0.007 ± 0.0003 |
| | 0.5 | 0.001 ± 0.00004 | 0.002 ± 0.0004 |
| | 1.0 | 0.001 ± 0.00003 | 0.002 ± 0.0002 |
| 3 | 0.5 | 0.001 ± 0.00008 | 0.003 ± 0.00007 |
| | 0.7 | — | 0.002 ± 0.00007 |
| | 1.0 | — | 0.001 ± 0.00001 |

* Enzyme activity (μmol min⁻¹ mL⁻¹) of the immobilized FGGsh155:CB[8] in different conditions. Measurements were performed using the CDNB/GSH substrate system in 20 mM KH₂PO₄, pH 6.5 and 7.0.

Kinetic analysis for GSH was performed using standard CDNB concentration (1 mM) and varying GSH concentrations. Correspondingly, standard concentration of GSH substrate (2.5 mM) and varying concentrations of CDNB were utilised in the kinetic analysis for CDNB.

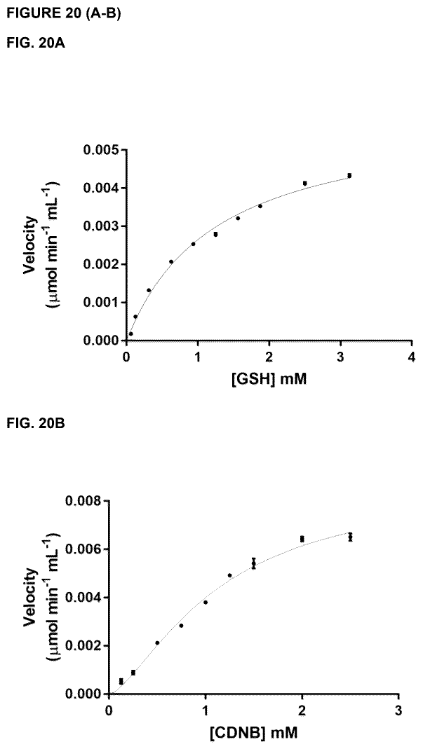

The kinetic parameters of the immobilized FGGsh155:CB[8] were calculated using the CDNB/GSH substrate system in 20 mM KH₂PO₄, PH 7.0, at 37° C. (FIG. 20) depicts the dependence of the enzyme's reaction rate on the substrate concentration. The kinetic constants Kₘ, S_{0.5} and k_{cat} are calculated for CDNB, with standard concentration of GSH (2.5 mM) and consecutive concentrations of CDNB. Respectively the kinetic analysis for GSH was carried with standard CDNB concentration (1 mM) and different GSH concentrations. The affinity constants Kₘ and S_{0.5} of the free and immobilized enzyme weren't significantly altered, however, the catalytic constant decreased substantially (Table 7). The reduction in k_{cat} is usually expected after immobilization (Neira & Herr, 2017). In addition, the enzyme exhibited enhanced positive synergy (Hill coefficient, nH: 1.47±0.12) after the immobilization, which might be linked to the modification of the enzyme's conformation during immobilization.

TABLE 7

| | | | | | k_{cat}/Kₘ | k_{cat}/S_{0.5} |
|---|---|---|---|---|---|---|
| | Kₘ | S_{0.5} | | | (min⁻¹ | (min⁻¹ |
| | (mM) | (mM) | | k_{cat} | mM⁻¹) | mM⁻¹) |
| | GSH | CDNB | n_{H} | (min⁻¹) | GSH | CDNB |
| FGGsh155:CB[8] | 1.43 ± 0.03 | 0.81 ± 0.05 | 1.01 ± 0.02 | 121 ± 1.14 | 84.62 | 149.38 |
| Immobilization | 1.26 ± 0.08 | 1.126 ± 0.12 | 1.47 ± 0.12 | 1.28 ± 0.12 | 1.01 | 1.14 |
| FGGsh155:CB[8] | | | | | | |

### Example 10: Kinetic Analysis and Inhibition Potency Induced by Butachlor

The half maximal inhibitory concentration (IC50), namely the inhibitor concentration that leads to 50% inhibition of the enzyme activity of FGGsh155:CB[8], was determined for constant substrate concentrations and varying butachlor concentrations. The utilised substrate concentrations were equal to the kinetic constant value of S_{0.5} for CDNB and 2.5 times the CDNB concentration for GSH. The correlation between the butachlor concentration from 0 to 600 nM (0 to 0.6 μM) and the remaining activity (%) of the immobilized enzyme was linear, thus standard curves for known herbicide concentrations were developed. The methodology was tested for drinking water samples derived from the Athens water supply network, as well as commercial mineral water (see Example 11). The results were analysed with the Graphpad Prism software.

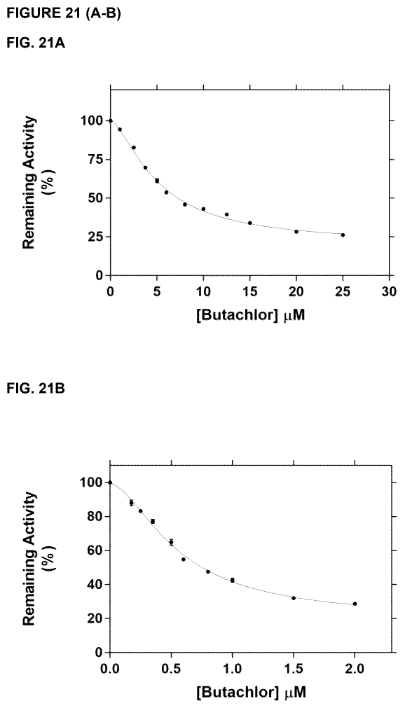

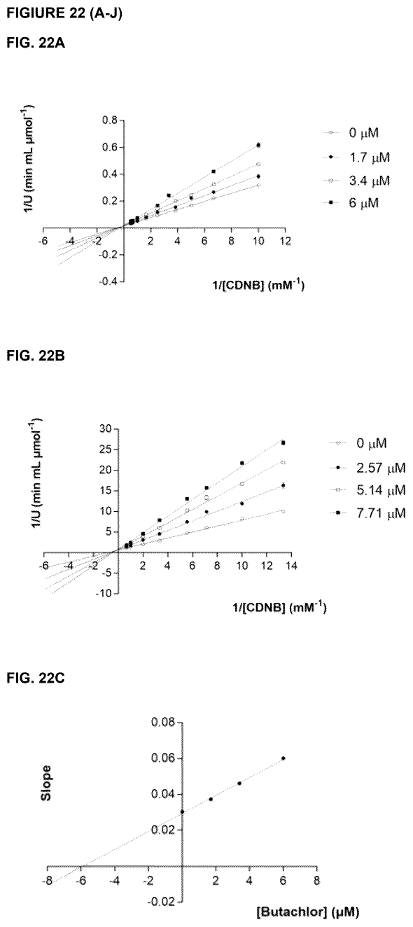

The experiments showed that the butachlor concentration that results to 50% decrease in enzyme activity was approximately ten times lower (0.56±0.02 μM) than that determined for the free enzyme (5.14±0.16 μM). Therefore, a ten-fold increase in sensitivity for butachlor was determined after immobilization on the chitosan carrier (FIG. 21).

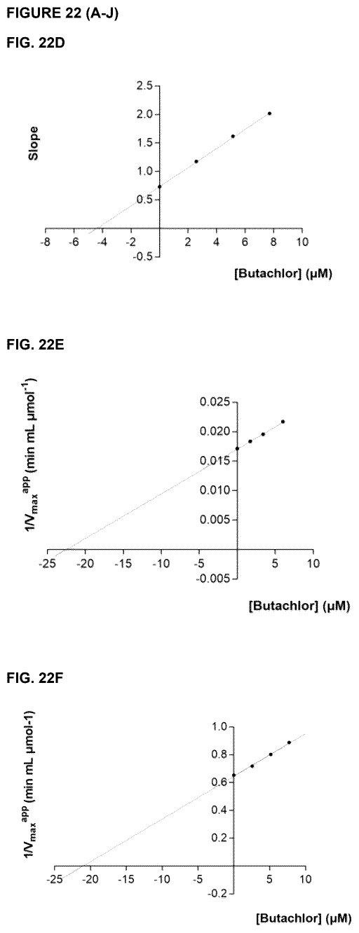

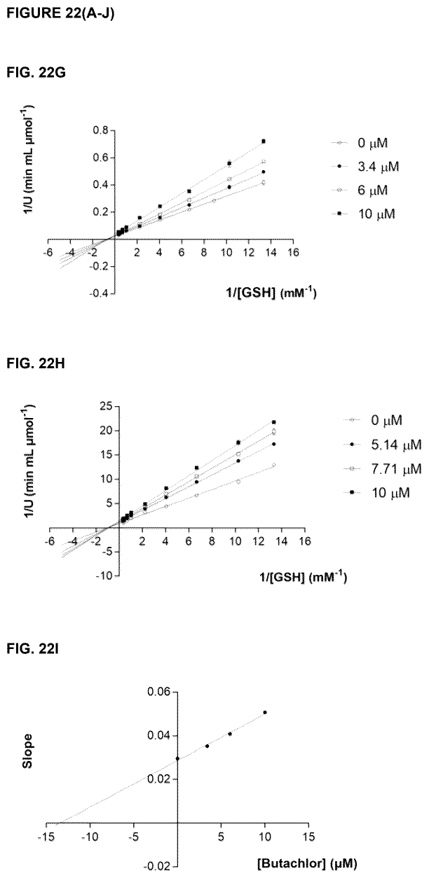

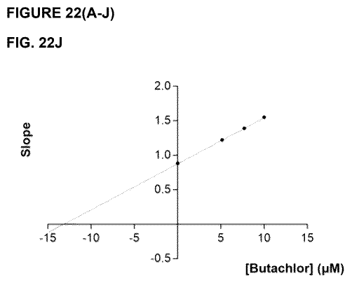

Kinetic analysis in presence of butachlor aimed to characterize of the type of inhibition, thus the binding site of the inhibitor, as well as to identify possible differences in kinetic behavior between the GST dimer form (sh155) and the hexamer FGGsh155:CB[8] that was used in the immobilization. Butachlor exhibited mixed type inhibition with Kᵢ<K_{i′} towards the CDNB substrate, which is considered a combination of the competitive and non-competitive inhibition pattern (FIG. 22). In the mixed inhibition pattern all of the kinetic constants (Kₘ, k_{cat}) change, and the inhibitor is able to bind to both the free enzyme (Kᵢ) and the enzyme-substrate complex (K_{i′}) with different affinities. Kᵢ and K_{i′} were determined from the secondary graphs of FIG. 22, at 5.92 μM (95% confidence interval from 4.57 to 7.82 μM, R²=0.997) and 22.55 μM (95% confidence interval from 19.65 to 26.29 μM, R²=0.998) respectively for sh155, and at 4,42 μM (95% confidence interval from 3.79 to 5.15 μM) and 21.1 μM (95% confidence interval from 17.16 to 26.81) for FGGsh155:CB[8]. Butachlor exhibited non-competitive inhibition towards GSH, since Kₘ remained constant but Vₘₐₓ and k_{cat} decreased as determined with kinetic analysis with increasing inhibitor concentration (Table 8, FIG. 22). This type of inhibition is a sub-case of the mixed inhibition pattern where Kᵢ=K_{i′}, i.e. the velocity of the reaction is decreased without affecting the affinity with the substrate. In this inhibition type, the inhibitor and the substrate bind independently at different enzyme sites. Kᵢ for sh155 was determined at 13.29 μM (95% confidence interval from 9.2 to 21.3 μM, R²=0.991) and at 13.21 μM (95% confidence interval from 12.42 to 14.07 μM, R²=0.999) for FGGsh155:CB[8]. The determined inhibition patterns as well as the inhibition constants for butachlor are consistent in both the dimer and hexamer enzyme forms, thus indicating that the conformation change did not affect the inhibition pattern induced by butachlor.

TABLE 8

| | Added butachlor | Found butachlor | Recovery |
|---|---|---|---|
| | (μM) | (μM) | (%) |
| Athens water | 0.05 | 0.046 ± 0.003 | 94.96 ± 4.3 |
| supply | 0.2 | 0.191 ± 0.001 | 96.16 ± 0.17 |
| network | 0.3 | 0.308 ± 0.008 | 103.12 ± 1.88 |
| | 0.4 | 0.416 ± 0.005 | 104.13 ± 0.87 |
| | 0.6 | 0.617 ± 0.003 | 102.55 ± 0.09 |
| Bottled | 0.1 | 0.095 ± 0.005 | 96.47 ± 1.89 |
| mineral water | 0.2 | 0.215 ± 0.005 | 106.86 ± 1.96 |
| | 0.3 | 0.307 ± 0.005 | 102.46 ± 1.55 |
| | 0.4 | 0.413 ± 0.012 | 103.49 ± 3.13 |
| | 0.55 | 0.56 ± 0.010 | 102 ± 1.25 |

* Recovery results of tap and bottled water samples spiked with known concentrations of butachlor. Found butachlor concentrations were calculated based on the standard reference curves. Results were obtained from at least three replicate measurements (Mean ± SD, N = 3).

### Example 11. Determination of Butachlor Concentrations in Water Samples

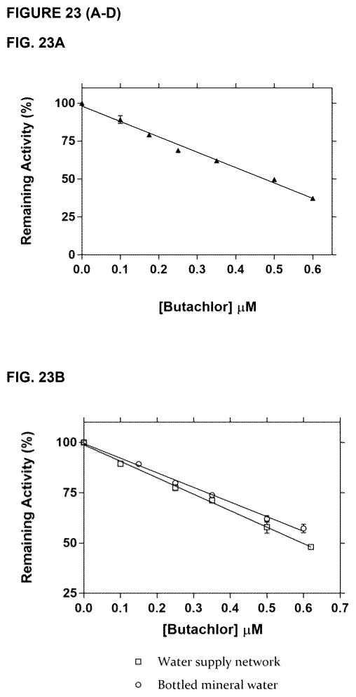

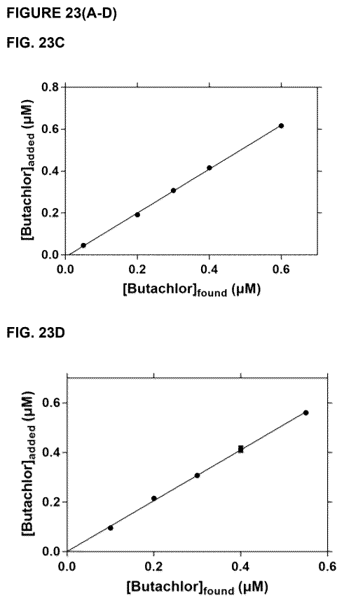

The basis for the detection of the herbicide butachlor in water samples was the determination of inhibition induced in the immobilized enzyme form. Initially, a linear correlation between the butachlor concentration from 0 to 600 nM and the remaining enzyme activity was observed in the graphs used for the determination of the IC50. The linear response of inhibition of the immobilized enzyme due to butachlor was described by the equation y=−101.3x+98.07, R²=0.991 (FIG. 23A). This observation led to the development of a methodology for determination of butachlor in drinking water samples based on the detection of inhibition as determined by a standard reference curve. Accordingly, standard curves were generated for spiked, with known concentrations of butachlor, samples of drinking water from the Athens' supply network (y=−81.84x+98.81, R²=0.997) and bottled mineral water (y=−72.69x+99.46, R²=0.995) (FIG. 23B). The detection range of butachlor with this methodology was determined up to 180 μg/L (600 nM) in water samples, with the acceptable amount of a pesticide in surface water at 1.0 μg/L and in drinking water at 0.1 μg/L (Council Directive 98/83/EC). The detection range up to 0.6 μM (180 μg/L) of the developed biosensor, with detection limit at approximately 31 μg/L for butachlor, achieves comparatively high sensitivity and selectivity.

The methodology was evaluated with recovery experiments of spiked drinking water samples, with known but random concentrations of butachlor within the range of the standard curve. Recovery of butachlor in the spiked drinking water from the supply network ranged between 94.96±4.3% and 104.13±0.87%, with a mean value of 100.19% and a standard deviation of 4.28% (N=5). Recoveries for the spiked mineral water samples ranged between 96.47 and 106.86, with a mean value of 102.26% and a standard deviation of 3.75% (N=5). The correlation between the added and found butachlor tested concentrations for network supply water (R² 0.9993, p<0.0001) and mineral water (R² 0.9987, p<0.0001) is depicted in FIG. 23C & 23D. The results show the reliability of the methodology for detection of butachlor.

### Example 12: Enzyme Stability of the Immobilized, Unmodified and Modified Enzyme Form of FGGsh155

Enzymatic stability was determined as the retention of enzyme activity overtime upon storage at 4° C. for the FGGsh155:CB[8] before and after immobilization, as well as the unmodified form of FGGsh155. Monitoring of the enzyme inactivation was determined using the CDNB/GSH substrate system.

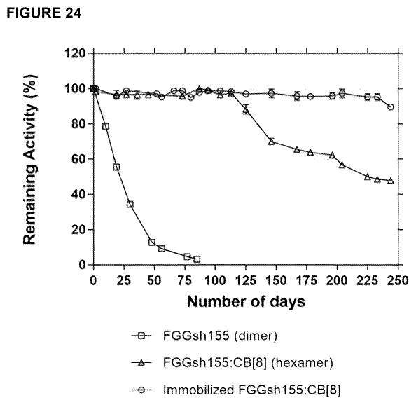

The hexamer enzyme form FGGsh155:CB[8], both before and after immobilisation, shows improved stability over time compared to the dimer FGGsh155 (FIG. 24). The dimer enzyme retains approximately 50% of its activity for the first twenty days of measurements. The decrease in enzyme activity was negligible for the free and immobilized form of the enzyme FGGsh155:CB[8] for the first 125 days of measurements. However, by day 244 the non-immobilized enzyme exhibited 47.9±0.23% remaining activity, yet the immobilised enzyme retained 89.56±0.24% of its activity. Therefore, immobilization of FGGsh155:CB[8] significantly contributed to the retention of the enzyme's activity, whilst rendering the enzyme reusable and thus reducing the cost of the method.

SEQUENCE TABLE

| SEQ | | |
|---|---|---|
| ID | | |
| NO | MOLECULE | SEQUENCE |
| 1 | GSTF sh101 | MFGGAPVKVFGPAMSTNVARVTLCLEEVGAEYEVVNIDFNTMEH |
| | | KSPEHLARNPFGQIPAFQDGDLLLWESRAISKYVLRKYKTDEVDL |
| | | LREGNLKEAAMVDVWTEYDAHTYNPALSPIVYECLINPLMRGLPT |
| | | NQTWVDESLEKLKKVLEVYEARLSQHKYLAGDFVSFADLNHFPYT |
| | | FYFMATPHAALFDSYPHVKAWWDRLMARPAVKKIAATMVPPKA |
| 2 | GSTF sh155 | MFGGAPVKVFGPAMSTNVARVTLCLEEVGAEYEVVDIDFKAMEH |
| | | KSPEHLVRNPFGQIPAFQDGDLLLFESRAIAKYVLRKYKTDEVDLL |
| | | REGNLKEAAMVDVWTEYDAHTYNPALSPIVYECLINPLMRGLPTN |
| | | QTWVDESLEKLKKVLEVYEARLSQHKYLAGDFVSFADLNHFPYTF |
| | | YFMATPHAALFDSYPHVKAWWDRLMARPAVKKIAATMVPPKA |
| 3 | primer | 5′ ATGTTTGGAGGAGCACCTGTAAAAGTCTTTGG 3′ |
| 4 | primer | 5′ ATGTATATCTCCTTCTTATAGTTAAAC 3′ |
| 5 | FGG tag | 5′ TTTGGAGGA 3′ |

1. A biosensor comprising a glutathione S-transferase homohexamer, wherein each transferase monomer of said homohexamer comprises a Phenylalanine-Glycine-Glycine tag at its N-terminal site, wherein said N-tagged monomer is assembled to a homohexamer through binding of Cucurbit[n]uril, preferably Cucurbit[8]uril, to said Phenylalanine-Glycine-Glycine tag and wherein said homohexamer is further characterised that it is immobilised on a polymer carrier.

2. The biosensor of claim 1, wherein said N-tagged monomer is assembled to an homohexamer through binding of Cucurbit[8]uril to said Phenylalanine-Glycine-Glycine tag and wherein the concentration ratio of said N-tagged monomer to Cucurbit[8]uril is 1:1.

3. The biosensor of claim 1 or 2, wherein each said glutathione S-transferase monomer comprises the SEQ ID NO: 1 or SEQ ID NO: 2.

4. The biosensor of any one of claims 1 to 3, wherein said homohexamer is immobilised on a glutaraldehyde-crosslinked chitosan carrier.

5. A method of production of the biosensor of any one of claims 1 to 4 characterised in that said method comprises the following steps of
mixing a chitosan gel with a glutaraldehyde solution so that a cross-linked chitosan carrier is formed
adding a solution of said glutathione-S-transferase homohexamer to said cross-linked chitosan carrier.

6. The method of claim 5 wherein said method is further characterised in that said method comprises the following steps of
dissolving 2%-4% (w/v) of chitosan in 2% (v/v) acetic acid so that a chitosan gel is formed
storing said chitosan gel for 12 to 24 hours at 4° C.
adding a glutaraldehyde solution at a concentration of 0.4%-1% (v/v), said glutaraldehyde being diluted in 20 mM KH₂PO₄, PH 7.0
washing with 2 mL 20 mM KH₂PO₄, PH 7
storing the cross-linked chitosan carrier for 3 to 10 days
adding a solution of glutathione S-transferase homohexamer at a concentration of 0.5 to 1 mg/mL
incubating said solution added to the carrier for 12 to 24 hours at around 25° C. with stirring at about 50-80 rpm
removing the non-absorbed solution of glutathione S-transferase homohexamer
incubating the carrier with a solution of 2 mg/mL NaBH₄ for about 30 min
washing with 20 mM KH₂PO₄, PH 7 in order to remove the unbound enzyme

7. A method of detecting a xenobiotic in a sample using the biosensor of any one of claims 1-4

8. A method according to claim 7, wherein said xenobiotic is a herbicide, preferably a chloroacetanilide herbicide, more preferably butachlor and wherein said sample is a water sample.

9. Use of the biosensor of any one of claims 1 to 4 for the detection of a xenobiotic, preferably a chloroacetanilide herbicide, more preferably butachlor, in a sample, preferably a water sample.

10. A kit comprising the biosensor of any one of claims 1 to 4 for the detection of a xenobiotic, preferably a chloroacetanilide herbicide, more preferably butachlor, in a sample.
